(19) United States
(12) Patent Application Publication
(10) Pub. No.: US 2014/0323488 A1
(43) Pub. Date: Oct. 30, 2014

(54) DISUBSTITUTED TRIAZINE DIMERS FOR TREATMENT AND/OR PREVENTION OF INFECTIOUS DISEASES

(71) Applicants: Wim LEWI, (US); Universiteit Antwerpen, Antwerpen (BE); Shakturana CV, Turnhout (BE)

(72) Inventors: Venkatraj Muthusamy, Wilrijk (BE); Louis Maes, Wilrijk (BE); Jan Heeres, Vosselaar (BE); Koen Augustyns, Wilrijk (BE); Pieter Van Der Veken, Wilrijk (BE); Jurgen Joossens, Wilrijk (BE); Paulas Joannes Lewi, Turnhout (BE)

(21) Appl. No.: 14/356,762

(22) PCT Filed: Nov. 9, 2012
(86) PCT No.: PCT/EP2012/072308

§ 371 Date: May 7, 2014

(30) Foreign Application Priority Data
Nov. 10, 2011 (GB) 1119358.8

(51) Int. Cl. C07D 403/12

(52) U.S. Cl. CPC C07D 403/12
USPC 514/245; 544/198; 544/209

(57) ABSTRACT

The present invention relates to novel compounds (I) containing two disubstituted triazine rings covalently linked by an organic linker, thereby creating dimers. These compounds show activity against the causative infective agents of infectious diseases such as African trypanosomiasis, Chagas disease, Leishmaniasis, Malaria and HIV. The invention further relates to the prevention and/or treatment of these diseases.

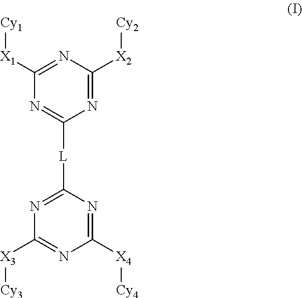

### FIELD OF THE INVENTION

The present invention relates to novel compounds containing two disubstituted triazine rings covalently linked by an organic linker, thereby creating dimers. These compounds show activity against the causative infective agents of infectious diseases such as African trypanosomiasis, Chagas disease, Leishmaniasis, Malaria and HIV. The invention further relates to the prevention and/or treatment of these diseases.

### BACKGROUND TO THE INVENTION

An infectious disease is a disease that is caused by the invasion of a host by pathogenic biological agents and can be transmitted to other individuals (that is, they are infectious). There are five major types of infectious agents: bacteria, viruses, fungi, protozoa, and helminths. The most common infectious diseases worldwide but not limited to HIV/AIDS, Tuberculosis, Malaria are African Trypanosomiasis (sleeping sickness), Cholera, Cryptosporidiosis, Dengue, Diarrhea, Hepatitis A, B, C, Influenza, Japanese Encephalitis, Leishmaniasis, Measles, Meningitis, Onchocerciasis (river blindness), Pneumonia, Rotavirus, Schistosomiasis, Shigellosis, Strep Throat, Typhoid and Yellow Fever. Infectious diseases kill more people worldwide than any other single cause. Approximately 15 million people die each year due to infectious diseases—nearly all live in developing countries. The top five causes of death from infectious disease are lower respiratory tract infections, HIV, diarrhoeal diseases, tuberculosis and malaria. Infectious diseases also account for over a quarter of the world's morbidity, as measured by disability-adjusted life years (DALYs). People infected with one infectious disease become more susceptible to other diseases. For example, people living with HIV/AIDS are more likely to become ill from tuberculosis or malaria. Because some neglected diseases share common risk factors, people are often infected with multiple neglected diseases at the same time.

We have unexpectedly found novel scaffolds which are able to selectively inhibit growth of different infectious pathogens. The compounds derived from these scaffolds, which show inhibitory activity on the growth of infectious pathogens, can be used as a preventive or therapeutic treatment of the infectious disease caused by that pathogen. Currently we have evaluated a library of compounds based on these scaffolds on different infectious pathogens (e.g. Trypanosoma, HIV, Plasmodium and Leishmania). We found very potent compounds with high specificity against a number of infectious pathogens. Intertestingly, we were able to obtain compounds based on these scaffolds which do not show cytotoxic effects in in vitro assays.

Human African trypanosomiasis (sleeping sickness) is a parasitic disease occurring in people and animals, and is caused by protozoa of the species Trypanosoma brucei and transmitted by the Tsetse fly. There are 3 sub-species of Trypanosoma brucei: Trypanosoma brucei gambiense (T. b. gambiense), Trypanosoma brucei rhodesiense (T. b. rhodesiense) and Trypanosoma brucei brucei (T. b. brucei). T. b. gambiense causes a slow onset chronic trypanosomiasis in humans, T. b. rhodesiense causes a fast onset acute trypanosomiasis in humans and T. b. brucei causes animal African trypanosomiasis. T. b. brucei is not human infective due to its susceptibility to lysis by the human apolipoprotein L1. However, as it shares many features with T. b. gambiense and T. b. rhodesiense it is a representative and frequently used model organism for human infections in laboratory and animal studies.

Chagas disease (American trypanosomiasis) is a parasitic disease caused by the flagellate protozoan Trypanosoma cruzi.

Leishmaniasis is a disease caused by protozoan parasites that belong to the genus Leishmania and is transmitted by the bite of certain species of sand fly. Cutaneous leishmaniasis is the most common form of leishmaniasis. Visceral leishmaniasis is a severe form in which the parasites have migrated to the vital organs.

Malaria is a parasitic disease which is caused by various species of Plasmodium protozoa. Malaria remains the single most devastating parasitic infections agent in the world, particularly in the developing and tropical world. Malaria infects hundreds of millions and kills roughly 2 million people each year. The high rate of mortality associated with malaria can be attributed to the increasing occurrence of Plasmodium falciparum, the most deadly of the four human infecting malarial parasites, to the contemporary antimalarial drugs.

Acquired immune deficiency syndrome (AIDS) is caused by the human immunodeficiency virus type-1 (HIV-1). HIV-1 non-nucleoside reverse transcriptase inhibitors (NNRTIs) are important in drug combination therapies (highly active antiretroviral therapy or HAART) currently used to treat HIV infection and AIDS due to their unique antiviral activity.

In addition, the overlap of Human Immunodeficiency Virus (HIV) with parasitic infections results in growing number of cases of dually infected individuals (1-3).

Diaryltriazines (DATA, monomers) are very potent NNRTIs and have anti-HIV-1 activity with nanomolar EC₅₀ values (4-9). Some of the most potent DATA monomers show an in vitro cytotoxic effect at 10 μM. We found that these monomers also exhibit antiparasitic activity but they are not highly potent and some of them are cytotoxic (Table 1). There are currently no satisfactory treatments available for neglected diseases. A need thus exists for an antiparasitic compound for humans that is more effective and less toxic than those currently available.

We identified novel scaffolds that contain two disubstituted triazine rings covalently linked by an organic linker. In most cases the compounds based on the scaffolds show an improved activity and/or less pronounced in vitro cytotoxic effects compared to the corresponding monomer compound. These two aspects make this invention an important improvement compared to the current state of the art. Moreover, the scaffolds have led to the discovery of a series of very potent and selective anti-viral and/or anti-parasitic compounds displaying no cytotoxic effects when evaluated in a broadly integrated anti-viral and anti-parasitic screen.

There is only one report of triazine dimers in WO2005049607 that has been disclosed for the treatment of autoimmune diseases. However, said reference is silent about the potential application of said compounds in the treatment and/or prevention of infectious diseases.

The present invention discloses compounds which differ from prior art compounds in structure and/or pharmacological activity. Surprisingly it has been found that these compounds are not only active against viral infections but also highly active against parasitic diseases with no cytotoxicity which could be very useful to treat patients with multiple infectious diseases at the same time.

### SUMMARY OF THE INVENTION

The invention is based on novel scaffolds, which contain two disubstituted triazine rings, which are covalently linked by an organic linker. Unexpectedly, the dimerized compounds of the invention still showed strong activity against HIV, while displaying very low cytotoxicity values. Surprisingly, the compounds not only showed activity against HIV, but were also potent inhibitors of Trypanosoma, Leishmania and Plasmodium, the infectious agents that cause human African trypanosomiasis, Chagas disease, Leishmaniasis and Malaria. It was even possible to design compounds with a less pronounced effect for HIV and a more specific effect on Trypanosomes.

Viewed from a first aspect, the invention provides a compound of formula I or a stereoisomer, tautomer, racemic, metabolite, pro- or predrug, salt, hydrate, or solvate thereof,

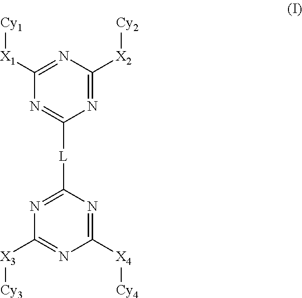

Wherein
X₁, X₂, X₃, and X₄ are independently selected from the group comprising —NRₐ—, —O—, —S—, and —CHR_{b}—;
Rₐ and R_{b} are each independently selected from H and alkyl;
Cy₁, Cy₂, Cy₃ and Cy₄ are independently selected from aryl, heteroaryl, and cycloalkyl; wherein said aryl, heteroaryl, and cycloalkyl are optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

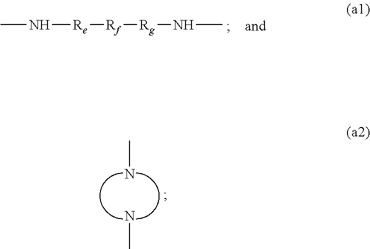

wherein subformula (a2) represents a 5- or 6-membered aromatic or non-aromatic heterocyclic group containing at least two nitrogen atoms;
Rₑ, and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond; and that at least one of Cy₁, Cy₂, Cy₃, or Cy₄, is substituted with two or more substituents,
provided that said compound is not
1,2-Ethanediamine, N,N′-bis[4,6-bis(2,6-dimethylphenoxy)-1,3,5-triazin-2-yl], or
Phenol,4,4′,4″,4′″-(1,2-ethanediylbis(imino-1,3,5-triazine-6,2,4-triylbis(thio)))tetrakis(2,6-bis(1,1-dimethyletyl))

Viewed from a further aspect, the invention provides a pharmaceutical and/or veterinary composition comprising a compound of the invention.

Viewed from a still further aspect, the invention provides a compound of the invention for use in human or veterinary medicine.

Viewed from a still further aspect, the invention provides the use of a compound of the invention in the preparation of a medicament for the prevention and/or treatment of an infectious disease.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As already mentioned hereinbefore, in a first aspect the present invention provides compounds of Formula I, including a stereoisomer, tautomer, racemic, metabolite, pro- or predrug, salt, hydrate, or solvate thereof

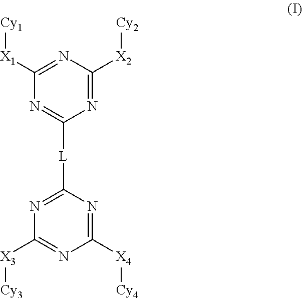

Wherein
X₁, X₂, X₃, and X₄ are independently selected from the group comprising —NRₐ—, —O—, —S—, and —CHR_{b}—;
Rₐ and R_{b} are each independently selected from H and alkyl;
Cy₁, Cy₃ and Cy₄ are independently selected from aryl, heteroaryl, and cycloalkyl; wherein said aryl, heteroaryl, and cycloalkyl are optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

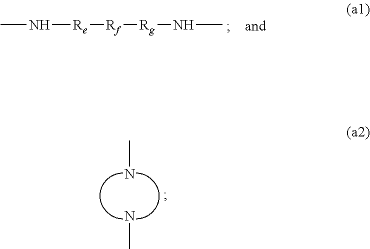

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond; and that at least one of Cy₁, Cy₂, Cy₃, or Cy₄, is substituted with two or more substituents,
provided that said compound is not
1,2-Ethanediamine, N,N′-bis[4,6-bis(2,6-dimethylphenoxy)-1,3,5-triazin-2-yl], or
Phenol,4,4′,4″,4′″-(1,2-ethanediylbis(imino-1,3,5-triazine-6,2,4-triylbis(thio)))tetrakis(2,6-bis(1,1-dimethyletyl))
This invention also provides compounds of formula I, including a stereoisomer, tautomer, racemic, metabolite, pro- or predrug, salt, hydrate, or solvate thereof, wherein one or more of the following applies:
X₁, X₂, X₃, and X₄ are independently selected from the group comprising —NRₐ—, —O—, —S—, and —CHR_{b}—;
Rₐ and R_{b} are each independently selected from H and alkyl;
Cy₁, Cy₂, Cy₃ and Cy₄ are independently selected from aryl, heteroaryl, and cycloalkyl; wherein said aryl, heteroaryl, and cycloalkyl are optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

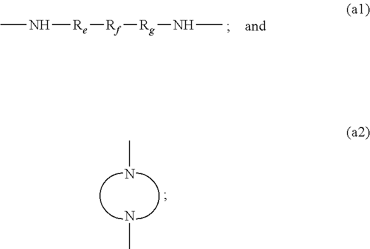

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond; and that at least one of Cy₁, Cy₂, Cy₃, or Cy₄, is substituted with two or more substituents,
provided that said compound is not
1,2-Ethanediamine,N,N′-bis[4,6-bis(2,6-dimethylphenoxy)-1,3,5-triazin-2-yl], or
Phenol,4,4′,4″,4′″-(1,2-ethanediylbis(imino-1,3,5-triazine-6,2,4-triylbis(thio)))tetrakis(2,6-bis(1,1-dimethyletyl))

When describing the compounds of the invention, the terms used are to be construed in accordance with the following definitions, unless a context dictates otherwise:

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 6 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers. C₁₋₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3-, or 4-methylcyclopentyl, cyclopentylmethylene, and cyclohexyl.

The term “alkenyl”, as used herein, unless otherwise indicated, means straight-chain, cyclic, or branched-chain hydrocarbon radicals containing at least one carbon-carbon double bond. Examples of alkenyl radicals include ethenyl, E- and Z-propenyl, isopropenyl, E- and Z-butenyl, E- and Z-isobutenyl, E- and Z-pentenyl, E- and Z-hexenyl, E,E-, E,Z-, Z,E-, Z,Z-hexadienyl, and the like. An optionally substituted alkenyl refers to an alkenyl having optionally one or more substituents (for example 1, 2, 3 or 4), selected from those defined above for substituted alkyl. The term “alkynyl”, as used herein, unless otherwise indicated, means straight-chain or branched-chain hydrocarbon radicals containing at least one carbon-carbon triple bond. Examples of alkynyl radicals include ethynyl, E- and Z-propynyl, isopropynyl, E- and Z-butynyl, E- and Z-isobutynyl, E- and Z-pentynyl, E, Z-hexynyl, and the like. An optionally substituted alkynyl refers to an alkynyl having optionally one or more substituents (for example 1, 2, 3 or 4), selected from those defined above for substituted alkyl.

The term “halo” or “halogen” as a group or part of a group is generic for fluoro, chloro, bromo, or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl, and the like.

The term “cycloalkyl” by itself or as part of another substituent is a cyclic alkyl group, that is to say, a monovalent, saturated, or unsaturated hydrocarbyl group having 1, 2, or 3 cyclic structure. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 15 atoms. The further rings of multi-ring cycloalkyls may be either fused, bridged and/or joined through one or more spiro atoms. Cycloalkyl groups may also be considered to be a subset of homocyclic rings discussed hereinafter. Examples of cycloalkyl groups include but are not limited to cyclopropyl, cyclobutyl, cyclopentyl, cyclohexyl, cycloheptyl, cyclooctyl, cyclononyl, adamantanyl, bicyclo(2.2.1)heptanyl and cyclodecyl with cyclopropyl, cyclopentyl, cyclohexyl, adamantanyl, and bicyclo(2.2.1)heptanyl being particularly preferred. An “optionally substituted cycloalkyl” refers to a cycloalkyl having optionally one or more substituents (for example 1 to 3 substituents, for example 1, 2, 3 or 4 substituents), selected from those defined above for substituted alkyl. When the suffix “ene” is used in conjunction with a cyclic group, hereinafter also referred to as “Cycloalkylene”, this is intended to mean the cyclic group as defined herein having two single bonds as points of attachment to other groups. Cycloalkylene groups of this invention preferably comprise the same number of carbon atoms as their cycloalkyl radical counterparts.

The terms “heterocyclyl” or “heterocyclo” as used herein by itself or as part of another group refer to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, phthalazinyl, oxetanyl, thietanyl, 3-dioxolanyl, 1,3-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene) or linked covalently, typically containing 6 to 10 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1-2-, 3-, 4-, or 5-acenaphtylenyl, 3-, 4-, or 5-acenaphtenyl, 1-, 2-, 3-, 4-, or 10-phenanthryl, 1- or 2-pentalenyl, 1, 2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, and 1-, 2-, 3-, 4-, or 5-pyrenyl.

Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, benzopyranyl, 1(4H)-benzopyranyl, 1(2H)-benzopyranyl, 3,4-dihydro-1(2H)-benzopyranyl, 3,4-dihydro-1(2H)-benzopyranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl, 7-azaindolyl, 6-azaindolyl, 5-azaindolyl, 4-azaindolyl.

The term “alkoxy” or “alkyloxy” as used herein refers to a radical having the Formula —OR^{b} wherein R^{b} is alkyl. Preferably, alkoxy is C₁-C₁₀ alkoxy, C₁-C₆ alkoxy, or C₁-C₄ alkoxy. Non-limiting examples of suitable alkoxy include methoxy, ethoxy, propoxy, isopropoxy, butoxy, isobutoxy, sec-butoxy, tert-butoxy, pentyloxy and hexyloxy. Where the oxygen atom in an alkoxy group is substituted with sulfur, the resultant radical is referred to as thioalkoxy. “Haloalkoxy” is an alkoxy group wherein one or more hydrogen atoms in the alkyl group are substituted with halogen. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy; trichloromethoxy, 2-bromoethoxy, pentafluoroethyl, 3,3,3-trichloropropoxy, 4,4,4-trichlorobutoxy.

The term “alkylamino” and ‘dialkylamino”, as used herein refers to an amino group substituted with one or two alkyl chain(s), respectively.

Whenever used in the present invention, the term ‘compounds of the invention’ or a similar term is meant to include the compounds of general Formula I or II and any subgroup thereof. This term also refers to a stereoisomer, tautomer, racemic, metabolite, pro- or predrug, salt, hydrate, or solvate thereof.

As used in the specification and the appended claims, the singular forms “a”, “an” and “the” include plural referents unless the context clearly dictates otherwise. By way of example, “a compound” means one compound or more than one compound.

In a further embodiment the invention provides a compound of formula I, wherein;
X₁, X₂, X₃, and X₄ are independently selected from the group comprising —NRₐ—, —O—, —S—, and —CHR_{b}—;
Rₐ and R_{b} are each independently selected from H and alkyl;
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl are optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

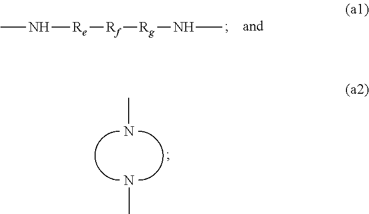

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond; and that at least one of Cy₁, Cy₂, Cy₃, or Cy₄, is substituted with two or more substituents;
   for use as a medicine.

In another further embodiment, the invention provides a compound according to the present invention wherein
L is selected from the groups with subformula

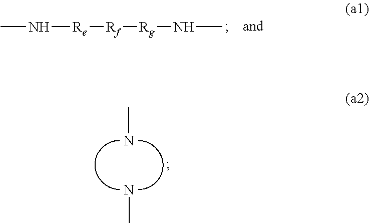

wherein subformula (a2) represents a 5- or 6-membered aromatic or non-aromatic heterocyclic group containing at least two nitrogen atoms; in particular a 6-membered group aromatic or non-aromatic group containing at least two nitrogen atoms; more in particular pyrazinylene or piperazinylene; and
R_{f} is a direct bond or selected from the group comprising aryl, heteroaryl, cycoalkyl, and heterocyclyl; in particular R_{f} is a direct bond or aryl; more in particular a direct bond or phenyl;

In another further embodiment, the invention provides a compound according to the present invention wherein
Rₐ and R_{b} are each independently selected from H and alkyl; in particular H and C₁₋₅alkyl; more in particular H and methyl;
Cy₁, Cy₂, Cy₃ and Cy₄ are each aryl optionally substituted with one or more substituents independently selected from the group comprising alkyl, alkoxy, halo, and cyano; in particular from the group comprising C₁₋₅alkyl, C₁₋₅alkoxy, halo, and cyano;
L is selected from the groups with subformula

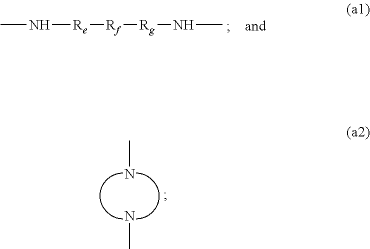

wherein subformula (a2) represents a 6-membered aromatic or non-aromatic heterocyclic group containing at least two nitrogen atoms; in particular pyrazinylene or piperazinylene; more in particular piperazinylene; and
Rₑ and R_{g} are independently a direct bond or alkyl; in particular a direct bond or C₁₋₅alkyl;
R_{f} is a direct bond or selected from the group comprising aryl, heteroaryl, cycoalkyl, and heterocyclyl; in particular R_{f} is a direct bond or aryl; more in particular R_{d} is a direct bond or phenyl;

In yet another embodiment, the present invention provides compounds of formula I wherein one or more of the following restrictions apply:
X₁, X₂, X₃, and X₄ are independently selected from the group comprising —NRₐ—, —O—, —S—, and —CHR_{b}—; in particular —NRₐ—, and —O—;
X₂ and X₄ are independently —NRₐ—, and X₁ and X₃ are —O—; in particular X₂ and X₄ are independently —NH—, and X₁ and X₃ are —O—;
X₁, X₂; X₃, and X₄ are independently —NRₐ—; in particular —NH—;
Rₐ and R_{b} are each independently selected from H and alkyl; in particular H and C₁₋₅alkyl; more in particular H and methyl;
Cy₁, Cy₂, Cy₃ and Cy₄ are independently selected from aryl, heteroaryl, and cycloalkyl; wherein said aryl, heteroaryl, and cycloalkyl are optionally substituted with one or more substituents selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-;
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl are optionally substituted with one or more substituents independently selected from the group comprising alkyl, alkoxy, halo, and cyano;
Cy₁, Cy₂, Cy₃ and Cy₄ is aryl optionally substituted with one or more substituents selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-;
Cy₁, Cy₂, Cy₃ and Cy₄ is aryl optionally substituted with one or more substituents independently selected from the group comprising alkyl, alkoxy, halo, and cyano; more in particular Cy₁, Cy₂, Cy₃ and Cy₄ is aryl optionally substituted with one or more substituents independently selected from the group comprising C₁₋₅alkyl, C₁₋₅alkoxy, halo, and cyano; and
L is selected from the groups with subformula

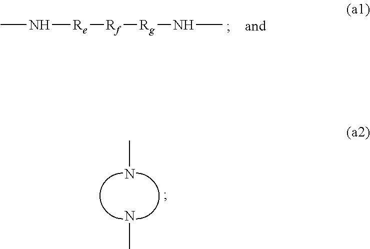

wherein subformula (a2) represents a 5- or 6-membered aromatic or non-aromatic heterocyclic group containing at least two nitrogen atoms; in particular a 6-membered group such as pyrazinylene or piperazinylene; more in particular piperazinylene;
Rₑ and R_{g} are independently a direct bond or alkyl; in particular a direct bond or C₁₋₅alkyl;
Rₑ and R_{g} are identical and selected from a direct bond or alkyl;
R_{f} is a direct bond or selected from the group comprising aryl, heteroaryl, cycoalkyl, and heterocyclyl; in particular R_{f} is a direct bond or aryl; more in particular R_{f} is a direct bond or phenyl;
Rₑ—R_{f}—R_{g} is C₁₋₁₀alkyl or aryl;
with the proviso that —Rₑ—R_{f}—R_{g}— is not a direct bond; and that at least one of Cy₁, Cy₂, Cy₃ or Cy₄, is substituted with two or more substituents.
   provided that said compound is not
1,2-Ethanediamine,N,N′-bis[4,6-bis(2,6-dimethylphenoxy)-1,3,5-triazin-2-yl], or
Phenol,4,4′,4″,4′″-(1,2-ethanediylbis(imino-1,3,5-triazine-6,2,4-triylbis(thio)))tetrakis(2,6-bis(1,1-dimethyletyl))

In another embodiment, the present invention provides compounds of formula II

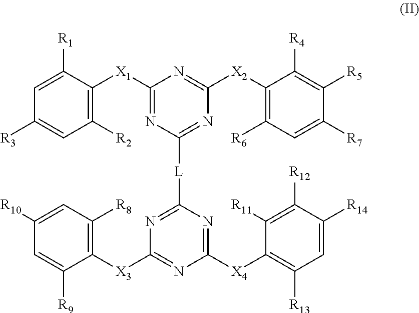

Wherein
X₁, X₂, X₃, and X₄ are independently selected from the group comprising —NRₐ—, —O—, —S—, and —CHR_{b}—;
Rₐ and R_{b} are each independently selected from H and alkyl;
R₁, R₂, R₃, R₄, R₅, R₆, R₇, R₈, R₉, R₁₀, R₁₁, R₁₂, R₁₃, and R₁₄ are independently selected from the group comprising H, alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

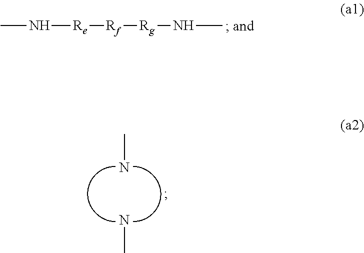

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that at least one of the phenyl rings is substituted with two or more substituents other than hydrogen; and that —Rₑ—R_{f}—R_{g}— is not a direct bond.
   provided that said compound is not
1,2-Ethanediamine, N,N′-bis[4,6-bis(2,6-dimethylphenoxy)-1,3,5-triazin-2-yl],

In a further embodiment, the present invention provides compounds of formula II wherein
X₁, X₂, X₃, and X₄ are independently selected from the group comprising —NRₐ—, —O—, —S—, and —CHR_{b}—; in particular —NRₐ—, and —O—;
Rₐ and R_{b} are each independently selected from H and alkyl; in particular H and C₁₋₅alkyl; more in particular H and methyl;
R₁, R₂, R₃, R₄, R₅, R₆, R₇, R₈, R₉, R₁₀, R₁₁, R₁₂, R₁₃, and R₁₄ are independently selected from the group comprising H, alkyl, alkoxy, halo, cyano, and —CH═CHCN; in particular from the group comprising H, C₁₋₅alkyl, C₁₋₅alkoxy, halo, and cyano; and
L is selected from the groups with subformula

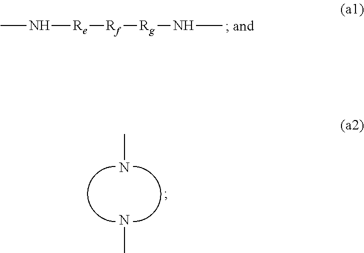

wherein subformula (a2) represents a 6-membered aromatic or non-aromatic heterocyclic group containing at least two nitrogen atoms; in particular pyrazinylene or piperazinylene; more in particular piperazinylene;
Rₑ and R_{g} are independently a direct bond or alkyl; in particular a direct bond or C₁₋₅alkyl;
R_{f} is a direct bond or selected from the group comprising aryl, heteroaryl, cycoalkyl, and heterocyclyl; in particular R_{f} is a direct bond or aryl; more in particular R_{d} is a direct bond or phenyl;
with the proviso that at least of one the phenyl rings is substituted with two or more substituents other than hydrogen; and that —Rₑ—R_{f}—R_{g}— is not a direct bond.
   provided that said compound is not
1,2-Ethanediamine,N,N′-bis[4,6-bis(2,6-dimethylphenoxy)-1,3,5-triazin-2-yl],

It is also an object of the present invention to provide compounds of formula II wherein one or more of the following restrictions apply:
X₁, X₂, X₃, and X₄ are independently selected from the group comprising —NRₐ—, —O—, —S—, and —CHR_{b}—; in particular —NRₐ—, and —O—;
X₂ and X₄ are independently —NRₐ—, and X₁ and X₃ are —O—; in particular X₂ and X₄ are independently —NH—, and X₁ and X₃ are —O—;
X₁, X₂, X₃, and X₄ are independently —NRₐ—; in particular —NH—;
Rₐ and R_{b} are each independently selected from H and alkyl; in particular H and C₁₋₅alkyl; more in particular H and methyl;
R₁, R₂, R₃, R₄, R₅, R₆, R₇, R₈, R₉, R₁₀, R₁₁, R₁₂, R₁₃, and R₁₄ are independently selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-
R₁, R₂, R₃, R₄, R₅, R₆, R₇, R₈, R₉, R₁₀, R₁₁, R₁₂, R₁₃, and R₁₄ are independently selected from the group comprising H, alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; in particular from the group comprising H, alkyl, alkoxy, halo, and cyano; more in particular from the group comprising H, C₁₋₅alkyl, C₁₋₅alkoxy, halo, and cyano; and
L is selected from the groups with subformula

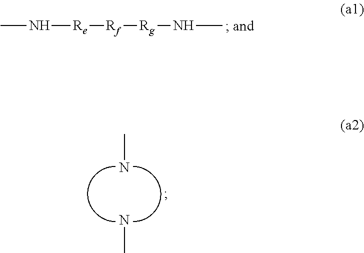

wherein subformula (a2) represents a 5- or 6-membered aromatic or non-aromatic heterocyclic group containing at least two nitrogen atoms; in particular a 6-membered group such as pyrazinylene or piperazinylene; more in particular piperazinylene;
Rₑ and R_{g} are independently a direct bond or alkyl; in particular a direct bond or C₁₋₅alkyl;
Rₑ and R_{g} are identical and selected from a direct bond or alkyl;
R_{f} is a direct bond or selected from the group comprising aryl, heteroaryl, cycoalkyl, and heterocyclyl; in particular R_{f} is a direct bond or aryl; more in particular R_{d} is a direct bond or phenyl;
Rₑ—R_{f}—R_{g} is C₁₋₁₀alkyl or aryl;
   with the proviso that at least of one the phenyl rings is substituted with two or more substituents other than hydrogen; and that —Rₑ—R_{f}—R_{g} is not a direct bond.
   provided that said compound is not
1,2-Ethanediamine,N,N′-bis[4,6-bis(2,6-dimethylphenoxy)-1,3,5-triazin-2-yl],

In a further embodiment, the invention provides a compound as described hereinbefore for use as a medicine.

In another further embodiment, the invention provides a compound as described hereinbefore for use as a human or veterinary medicine.

In a particular embodiment, the invention provides a compound of formula I or a stereoisomer, tautomer, racemic, metabolite, pro- or predrug, salt, hydrate, or solvate thereof,

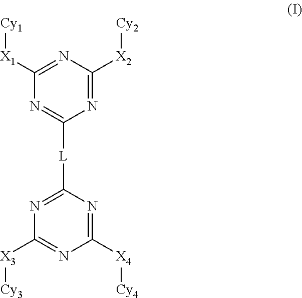

Wherein
X₁, X₂, X₃, and X₄ are independently selected from the group comprising —NRₐ—, —O—, —S—, and —CHR_{b}—;
Rₐ and R_{b} are each independently selected from H and alkyl;
Cy₁, Cy₂, Cy₃ and Cy₄ are independently selected from aryl, heteroaryl, cycloalkyl and heterocyclyl; wherein said aryl, heteroaryl, cycloalkyl and heterocyclyl are optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

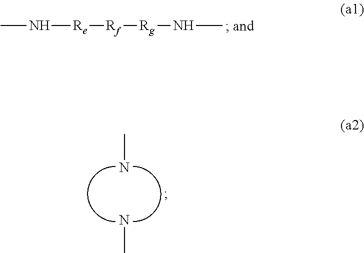

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
   with the proviso that —Rₑ—R_{f}—R_{g}— is not a direct bond;
   for use in the prevention and/or treatment of an infectious disease.

In yet another embodiment, the present invention provides compounds of formula I wherein one or more of the following restrictions apply:
X₁, X₂, X₃, and X₄ are independently selected from the group comprising —NRₐ—, —O—, —S—, and —CHR_{b}—; in particular —NRₐ—, and —O—;
X₂ and X₄ are independently —NRₐ—, and X₁ and X₃ are —O—; in particular X₂ and X₄ are independently —NH—, and X₁ and X₃ are —O—;
X₁, X₂, X₃, and X₄ are independently —NRₐ—; in particular —NH—;
Rₐ and R_{b} are each independently selected from H and alkyl; in particular H and C₁₋₅alkyl; more in particular H and methyl;
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl are optionally substituted with one or more substituents selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl are optionally substituted with one or more substituents independently selected from the group comprising alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; in particular Cy₂, Cy₃ and Cy₄ are independently selected from aryl, heteroaryl, cycloalkyl, and heterocyclyl; wherein said aryl, heteroaryl, cycloalkyl, and heterocyclyl are optionally substituted with one or more substituents independently selected from the group comprising alkyl, alkoxy, halo, and cyano;
Cy₁, Cy₂, Cy₃ and Cy₄ is aryl optionally substituted with one or more substituents independently selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-
Cy₁, Cy₂, Cy₃ and Cy₄ is aryl optionally substituted with one or more substituents independently selected from the group comprising alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; in particular Cy₁, Cy₂, Cy₃ and Cy₄ is aryl optionally substituted with one or more substituents independently selected from the group comprising alkyl, alkoxy, halo, and cyano; more in particular Cy₁, Cy₂, Cy₃ and Cy₄ is aryl optionally substituted with one or more substituents independently selected from the group comprising C₁₋₅alkyl, C₁₋₅alkoxy, halo, and cyano; and
L is selected from the groups with subformula

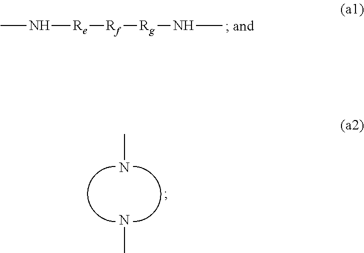

wherein subformula (a2) represents a 5- or 6-membered aromatic or non-aromatic heterocyclic group containing at least two nitrogen atoms; in particular a 6-membered group such as pyrazinylene or piperazinylene; more in particular piperazinylene;
Rₑ and R_{g} are independently a direct bond or alkyl; in particular a direct bond or C₁₋₅alkyl;
Rₑ and R_{g} are identical and selected from a direct bond or alkyl;
R_{f} is a direct bond or selected from the group comprising aryl, heteroaryl, cycoalkyl, and heterocyclyl; in particular R_{f} is a direct bond or aryl; more in particular R_{f} is a direct bond or phenyl;
Rₑ—R_{f}—R_{g} is C₁₋₁₀alkyl or aryl;
   with the proviso that —Rₑ—R_{f}—R_{g}— is not a direct bond;
   for use in the prevention and/or treatment of an infectious disease.

In another embodiment, the present invention provides compounds of formula II

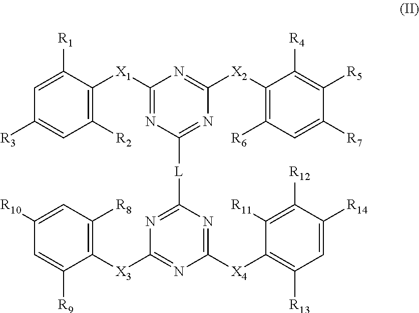

### Wherein

X₁, X₂, X₃, and X₄ are independently selected from the group comprising —NRₐ—, —O—, —S—, and —CHR_{b}—;
Rₐ and R_{b} are each independently selected from H and alkyl;
R₁, R₂, R₃, R₄, R₅, R₆, R₇, R₈, R₉, R₁₀, R₁₁, R₁₂, R₁₃, and R₁₄ are independently selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

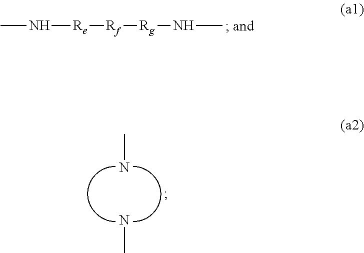

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond;
for use in the prevention and/or treatment of an infectious disease.

It is also an object of the present invention to provide compounds of formula II wherein one or more of the following restrictions apply:
X₁, X₂, X₃, and X₄ are independently selected from the group comprising —NRₐ—, —O—, —S—, and —CHR_{b}—; in particular —NRₐ—, and —O—;
X₂ and X₄ are independently —NRₐ—, and X₁ and X₃ are —O—; in particular X₂ and X₄ are independently —NH—, and X₁ and X₃ are —O—;
X₁, X₂, X₃, and X₄ are independently —NRₐ—; in particular —NH—;
Rₐ and R_{b} are each independently selected from H and alkyl; in particular H and C₁₋₅alkyl; more in particular H and methyl;
R₁, R₂, R₃, R₄, R₅, R₆, R₇, R₈, R₉, R₁₀, R₁₁, R₁₂, R₁₃, and R₁₄ are independently selected from the group comprising H, alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; in particular from the group comprising H, alkyl, alkoxy, halo, and cyano; more in particular from the group comprising H, C₁₋₅alkyl, C₁₋₅alkoxy, halo, and cyano; and
R₁, R₂, R₃, R₄, R₅, R₆, R₇, R₈, R₉, R₁₀, R₁₁, R₁₂, R₁₃, and R₁₄ are independently selected from -Alk and —R_{c};
each -Alk is independently selected from alkyl, alkenyl, and alkynyl; wherein said alkyl, alkenyl, and alkynyl is optionally substituted with one or more —R_{d};
each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-
L is selected from the groups with subformula

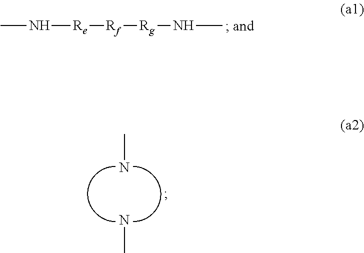

wherein subformula (a2) represents a 5- or 6-membered aromatic or non-aromatic heterocyclic group containing at least two nitrogen atoms; in particular a 6-membered group such as pyrazinylene or piperazinylene; more in particular piperazinylene;
Rₑ and R_{g} are independently a direct bond or alkyl; in particular a direct bond or C₁₋₅alkyl;
Rₑ and R_{g} are identical and selected from a direct bond or alkyl;
R_{f} is a direct bond or selected from the group comprising aryl, heteroaryl, cycoalkyl, and heterocyclyl; in particular R_{f} is a direct bond or aryl; more in particular R_{f} is a direct bond or phenyl;
Rₑ—R_{f}—R_{g} is C₁₋₁₀alkyl or aryl;
   with the proviso that —Rₑ—R_{f}—R_{g}— is not a direct bond;
   for use in the prevention and/or treatment of an infectious disease.

In a particular embodiment, the invention provides a compound according to the invention, or a composition comprising such a compound, for use in the prevention and/or treatment of an infectious disease.

In a further particular embodiment, the invention provides a compound according to the invention, or a composition comprising such a compound, for use in the prevention and/or treatment of a parasitic or viral disease. In a particular embodiment, the invention provides a compound according to the invention, or a composition comprising such a compound, for use in the prevention and/or treatment of a parasitic disease; more in particular a compound according to any one of formulas I or II hereinbefore, wherein X₁ and X₃ represent —O—; even more in particular a compound according to any one of formulas I or II hereinbefore, wherein X₁ and X₃ represent —O— and X₂ and X₄ are independently —NRₐ—; In another particular embodiment, the invention provides a compound according to the invention, or a composition comprising such a compound, for use in the prevention and/or treatment of a viral disease; more in particular a compound according to any one of formulas I or II hereinbefore, wherein X₁ and X₃ represent —NH—; even more in particular a compound according to any one of formulas I or II hereinbefore, wherein X₁ and X₃ represent —NH— and X₂ and X₄ are independently —NRₐ—.

Accordingly, it is an object of the present invention to provide a compound of formula I or a stereoisomer, tautomer, racemic, metabolite, pro- or predrug, salt, hydrate, or solvate thereof,

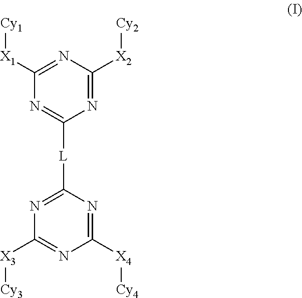

### Wherein

X₁ and X₃ are —O— and X₂ and X₄ are independently —NRₐ—;
Rₐ independently selected from H and alkyl;
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl is optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

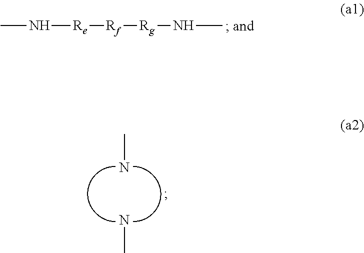

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond;
for use in the prevention and/or treatment of a parasitic disease.

Accordingly, it is an object of the present invention to provide a compound of formula I or a stereoisomer, tautomer, racemic, metabolite, pro- or predrug, salt, hydrate, or solvate thereof,

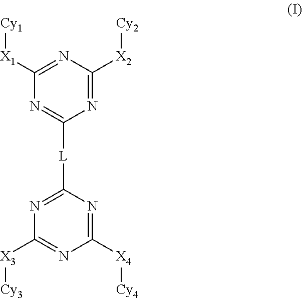

### Wherein

X₁, and X₃ are —NH— and X₂ and X₄ are independently —NRₐ—;
Rₐ independently selected from H and alkyl;
Cy₁, Cy₂, Cy₃ and Cy₄ are independently selected from aryl, heteroaryl, cycloalkyl, and heterocyclyl; wherein said aryl, heteroaryl, cycloalkyl, and heterocyclyl is optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group comprising haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula

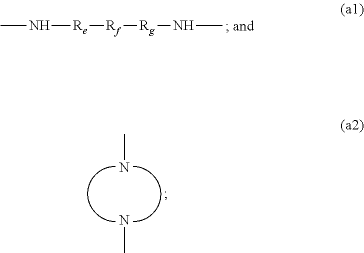

wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group comprising aryl, heteroaryl, cycoalkyl, and heterocyclyl;
with the proviso that —Rₑ—R_{f}—R_{g}— is not a direct bond;
for use in the prevention and/or treatment of a viral disease.

In particular, the invention provides a compound according to the invention, or a composition comprising such a compound, for use in the prevention and/or treatment of a disease associated with Trypanosoma, Leishmania, Plasmodium, or human immunodeficiency virus (HIV).

In a further embodiment, the present invention provides the use of a compound according to the invention, or a composition comprising such a compound, for use in the prevention and/or treatment of a disease associated with Trypanosoma. In particular a disease associated with Trypanosome selected from Chagas disease and African trypanosomiasis; more in particular Chagas disease and human African trypanosomiasis. In another particular embodiment, the invention provides the use of a compound according to the invention, or a composition comprising such a compound, for use in the prevention and/or treatment of a disease associated with Trypanosome brucei; in particular one or more Trypanosoma brucei strains selected from Trypanosome brucei gambiense, Trypanosoma brucei rhodesiense, and Trypanosome brucei brucei; more in particular selected from Trypanosoma brucei brucei and Trypanosoma brucei rhodesiense.

In another further embodiment, the invention provides the use of a compound according to the invention, or a composition comprising such a compound, for use in the prevention and/or treatment of a disease associated with Leishmania; in particular one or more Leishmania strains selected from Leishmania major, Leishmania tropica, Leishmania aethiopica, Leishmania Mexicana, Leishmania braziliensis, Leishmania donovani, and Leishmania infantum; more in particular Leishmania infantum. In particular the disease associated with Leishmania is leishmaniasis, including cutaneous leishmaniasis, mucocutaneous leishmaniasis, and visceral leishmaniasis.

In another further embodiment, the invention provides the use of a compound according to the invention, or a composition comprising such a compound, for use in the prevention and/or treatment of a disease associated with Plasmodium; more in particular one or more Plasmodium species selected from Plasmodium falciparum, Plasmodium vivax, Plasmodium malariae, Plasmodium knowlesi; more in particular Plasmodium falciparum.

In a further particular embodiment, the invention provides a compound according to the invention, or a composition comprising such a compound, for use in the prevention and/or treatment of a parasitic or viral disease. In particular, the invention provides a compound according to the invention, or a composition comprising such a compound, for use in the prevention and/or treatment of a disease associated with human immunodeficiency virus (HIV); in particular HIV type 1 and HIV type 2; more in particular HIV type 1.

The invention further provides a method for the prevention and/or treatment of an infectious disease; said method comprising administering to a subject in need thereof a therapeutic effective amount of a compound or a composition as defined herein.

In a preferred embodiment, the invention provides a method for the prevention and/or treatment of a disease associated with Trypanosoma; said method comprising administering to a subject in need thereof a therapeutic effective amount of a compound or a composition as defined herein.

In another preferred embodiment, the invention provides a method for the prevention and/or treatment of a disease associated with Leishmania; said method comprising administering to a subject in need thereof a therapeutic effective amount of a compound or a composition as defined herein.

In yet another preferred embodiment, the invention provides a method for the prevention and/or treatment of a disease associated with Plasmodium; said method comprising administering to a subject in need thereof a therapeutic effective amount of a compound or a composition as defined herein.

In another preferred embodiment, the invention provides a method for the prevention and/or treatment of a disease associated with human immunodeficiency virus (HIV); said method comprising administering to a subject in need thereof a therapeutic effective amount of a compound or a composition as defined herein.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

Generally, the compounds of this invention may be formulated as a pharmaceutical preparation or pharmaceutical composition comprising at least one compound of the invention and at least one pharmaceutically acceptable carrier, diluent or excipient and/or adjuvant.

By means of non-limiting examples, such a formulation may be in a form suitable for oral administration, parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives.

In addition, co-solvents such as alcohols may improve the solubility and/or the stability of the compounds. In the preparation of aqueous compositions, addition of salts of the compounds of the invention can be more suitable due to their increased water solubility.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

It should be noted that the compounds of the present invention can be administered as individual active ingredients, or as mixtures of several embodiments of the invention. The compounds of the invention may be used as single therapeutic agents or in combination with other therapeutic agents. Drugs that could be usefully combined with the compounds of the invention include other agents that treat and/or prevent infectious diseases, and/or agents that treat and/or prevent symptoms arising from an infectious disease.

The compounds can be administered by a variety of routes including the oral, rectal, vaginal, ocular, transdermal, subcutaneous, intravenous, intramuscular or intranasal routes, depending mainly on the specific preparation used and the condition to be treated or prevented, and with oral, rectal, vaginal and intravenous administration usually being preferred. The at least one compound of the invention will generally be administered in an “effective amount”, by which is meant any amount of a compound of the Formula I or II, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to US-A-6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly. As a non-limiting example, said pharmaceutical compositions can be administered orally or parentally for pre- and post-exposure prevention.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art. When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringers solution or isotonic sodium chloride solution, or suitable dispersing or wetting and suspending agents, such as sterile, bland, fixed oils, including synthetic mono- or diglycerides, and fatty acids, including oleic acid.

When rectally or vaginally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug. Other vaginal administration forms that are well-known to a skilled person include gels, films, sponges and intravaginal rings.

The compositions are also of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention and at least one suitable carrier (i.e. a carrier suitable for veterinary use). The invention also relates to the use of a compound of the invention in the preparation of such a composition.

As used herein, the term “infectious disease” is meant to comprise any disease or disorder resulting from the infection, presence and growth of a pathogenic biological agent in a host organism. For example, infectious pathogenic biological agents include some viruses, bacteria, fungi, protozoa, and multicellular parasites. Specific non-limiting examples of infectious diseases include AIDS; Respiratory tract infections; Tuberculosis; Malaria; Measles; Pertussis; Tetanus; Meningitis; Syphilis; Hepatitis A, B, C, and D; Chagas disease; Dengue; African trypanosomiasis; Leishmaniasis; Leprosy; Lymphatic filariasis; Onchocerciasis; and Schistosomiasis, Avian Influenza A (H5N1), Anthrax, Bluetongue, Dermatophytosis, Mycobacteriosis, Plague, West Nile Virus, Rabies, Lyme Disease, Hantavirus, Bovine Spongiform Encephalitis (“Mad Cow Disease”), Leptospirosis, and E. Coli.

The compounds of the present invention can be prepared according to the reaction schemes provided in the examples hereinafter, but those skilled in the art will appreciate that these are only illustrative for the invention and that the compounds of this invention can be prepared by any of several standard synthetic processes commonly used by those skilled in the art of organic chemistry.

The invention will now be illustrated by means of the following synthetic and biological examples, which do not limit the scope of the invention in any way.

### EXAMPLES

### Example 1

### Specific Examples of Compounds According to the Invention

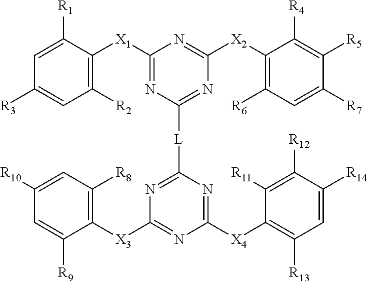

| Cpd | R₁ | R₂ | R₃ | X₁ | X₂ | R₄ | R₅ | R₆ | R₇ | Linker | R₈ | R₉ | R₁₀ | X₃ | X₄ | R₁₁ | R₁₂ | R₁₃ | R₁₄ |
|---|---|---|---|---|---|---|---|---|---|---|---|---|---|---|---|---|---|---|---|
| T1 | Me | Me | Me | NH | NH | Me | H | Me | Me | HN(CH₂)₂NH | Me | Me | Me | NH | NH | Me | H | Me | Me |
| T2 | Me | Me | Me | NH | NH | Me | H | Me | Me | HN(CH₂)₃NH | Me | Me | Me | NH | NH | Me | H | Me | Me |
| T3 | Me | Me | Me | NH | NH | Me | H | Me | Me | HN(CH₂)₄NH | Me | Me | Me | NH | NH | Me | H | Me | Me |
| T4 | Me | Me | Me | NH | NH | Me | H | Me | Me | HN(CH₂)₅NH | Me | Me | Me | NH | NH | Me | H | Me | Me |
| T5 | Me | Me | Me | NH | NH | H | H | H | CN | HN(CH₂)₂NH | Me | Me | Me | NH | NH | H | H | H | CN |
| T6 | Me | Me | Me | NH | NH | H | H | H | CN | HN(CH₂)₃NH | Me | Me | Me | NH | NH | H | H | H | CN |
| T7 | Me | Me | Me | NH | NH | H | H | H | CN | HN(CH₂)₄NH | Me | Me | Me | NH | NH | H | H | H | CN |
| T8 | Me | Me | Me | NH | NH | H | H | H | CN | HN(CH₂)₅NH | Me | Me | Me | NH | NH | H | H | H | CN |
| T9 | Me | Me | Me | NH | NH | H | H | H | CN | HN(CH₂)₆NH | Me | Me | Me | NH | NH | H | H | H | CN |
| T10 | Me | Me | Me | NH | NH | H | H | H | CN | HN(CH₂)₁₀NH | Me | Me | Me | NH | NH | H | H | H | CN |
| T11 | Me | Me | Me | NH | NH | H | H | H | CN | 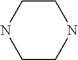 | Me | Me | Me | NH | NH | H | H | H | CN |
| T12 | Me | Me | Me | NH | NH | H | H | H | CN | 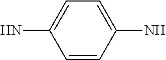 | Me | Me | Me | NH | NH | H | H | H | CN |
| T13 | Br | Br | Me | NH | NH | H | H | H | CN | HN(CH₂)₃NH | Br | Br | Me | NH | NH | H | H | H | CN |
| T14 | Br | Br | Me | NH | NH | H | H | H | CN | HN(CH₂)₄NH | Br | Br | Me | NH | NH | H | H | H | CN |
| T15 | Br | Br | Me | NH | NH | H | H | H | CN | HN(CH₂)₅NH | Br | Br | Me | NH | NH | H | H | H | CN |
| T16 | Br | H | Me | NH | NH | H | H | H | CN | HN(CH₂)₂NH | Br | H | Me | NH | NH | H | H | H | CN |
| T17 | Br | H | Me | NH | NH | H | H | H | CN | HN(CH₂)₃NH | Br | H | Me | NH | NH | H | H | H | CN |
| T18 | Br | H | Me | NH | NH | H | H | H | CN | HN(CH₂)₅NH | Br | H | Me | NH | NH | H | H | H | CN |
| T19 | Me | Me | H | NH | NH | H | H | H | CN | HN(CH₂)₂NH | Me | Me | H | NH | NH | H | H | H | CN |
| T20 | Me | Me | H | NH | NH | H | H | H | CN | HN(CH₂)₃NH | Me | Me | H | NH | NH | H | H | H | CN |
| T21 | Me | Me | H | NH | NH | H | H | H | CN | HN(CH₂)₅NH | Me | Me | H | NH | NH | H | H | H | CN |
| T22 | Me | Me | Br | NH | NH | H | H | H | CN | HN(CH₂)₂NH | Me | Me | Br | NH | NH | H | H | H | CN |
| T23 | Me | Me | Br | NH | NH | H | H | H | CN | HN(CH₂)₃NH | Me | Me | Br | NH | NH | H | H | H | CN |
| T24 | Me | Me | Br | NH | NH | H | H | H | CN | HN(CH₂)₅NH | Me | Me | Br | NH | NH | H | H | H | CN |
| T25 | Me | Me | Me | NH | NH | H | H | H | CN | HN(CH₂)₂NH | Br | Br | Me | NH | NH | H | H | H | CN |
| T26 | Me | Me | Me | NH | NH | H | H | H | CN | 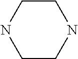 | Br | Br | Me | NH | NH | H | H | H | CN |
| T27 | Me | Me | Me | NH | NH | Me | H | Me | Me | HN(CH₂)₂NH | Me | Me | Me | NH | NH | H | H | H | CN |
| T28 | Me | Me | Me | NH | NH | H | H | Me | Me | HN(CH₂)₃NH | Me | Me | Me | NH | NH | H | H | H | CN |
| T29 | Me | Me | Me | NMe | NH | H | H | H | CN | HN(CH₂)₂NH | Me | Me | Me | NMe | NH | H | H | H | CN |
| T30 | Me | Me | Me | NMe | NH | H | H | H | CN | HN(CH₂)₃NH | Me | Me | Me | NMe | NH | H | H | H | CN |
| T31 | Me | Me | Me | O | NH | H | H | H | CN | HN(CH₂)₂NH | Me | Me | Me | O | NH | H | H | H | CN |
| T32 | Me | Me | Me | O | NH | H | H | H | CN | HN(CH₂)₃NH | Me | Me | Me | O | NH | H | H | H | CN |
| T33 | Me | Me | Me | O | NH | H | H | H | CN | HN(CH₂)₅NH | Me | Me | Me | O | NH | H | H | H | CN |
| T34 | Me | Me | Me | O | NH | H | H | H | H | HN(CH₂)₂NH | Me | Me | Me | O | NH | H | H | H | H |
| T35 | Me | Me | Me | O | NH | H | CN | H | H | HN(CH₂)₂NH | Me | Me | Me | O | NH | H | CN | H | H |
| T36 | Me | Me | Me | O | NH | H | H | H | H | HN(CH₂)₂NH | Me | Me | Me | O | NH | H | H | H | CN |
| T37 | Me | Me | Me | O | NH | H | CN | H | H | HN(CH₂)₂NH | Me | Me | Me | O | NH | H | H | H | CN |
| T38 | Me | Me | H | O | NH | H | H | H | CN | HN(CH₂)₂NH | Me | Me | H | O | NH | H | H | H | CN |
| T39 | Me | Me | H | O | NH | H | H | H | CN | HN(CH₂)₃NH | Me | Me | H | O | NH | H | H | H | CN |
| T40 | Me | Me | Cl | O | NH | H | H | H | CN | HN(CH₂)₂NH | Me | Me | Cl | O | NH | H | H | H | CN |
| T41 | Me | Me | Cl | O | NH | H | H | H | CN | HN(CH₂)₃NH | Me | Me | Cl | O | NH | H | H | H | CN |
| T42 | Me | Me | Br | O | NH | H | H | H | CN | HN(CH₂)₂NH | Me | Me | Br | O | NH | H | H | H | CN |
| T43 | Me | Me | Br | O | NH | H | H | H | CN | HN(CH₂)₃NH | Me | Me | Br | O | NH | H | H | H | CN |
| T44 | OMe | Me | Me | O | NH | H | H | H | CN | HN(CH₂)₂NH | OMe | OMe | Me | O | NH | H | H | H | CN |
| T45 | OMe | Me | Me | O | NH | H | H | H | CN | HN(CH₂)₃NH | OMe | OMe | Me | O | NH | H | H | H | CN |
| T46 | Me | Me | Me | NH | NH | H | H | H | CN | HN(CH₂)₂N | Me | Me | Me | O | NH | H | H | H | CN |

### Example 2

### Synthesis of Intermediates I1-I2 and Target Compounds T1-T4

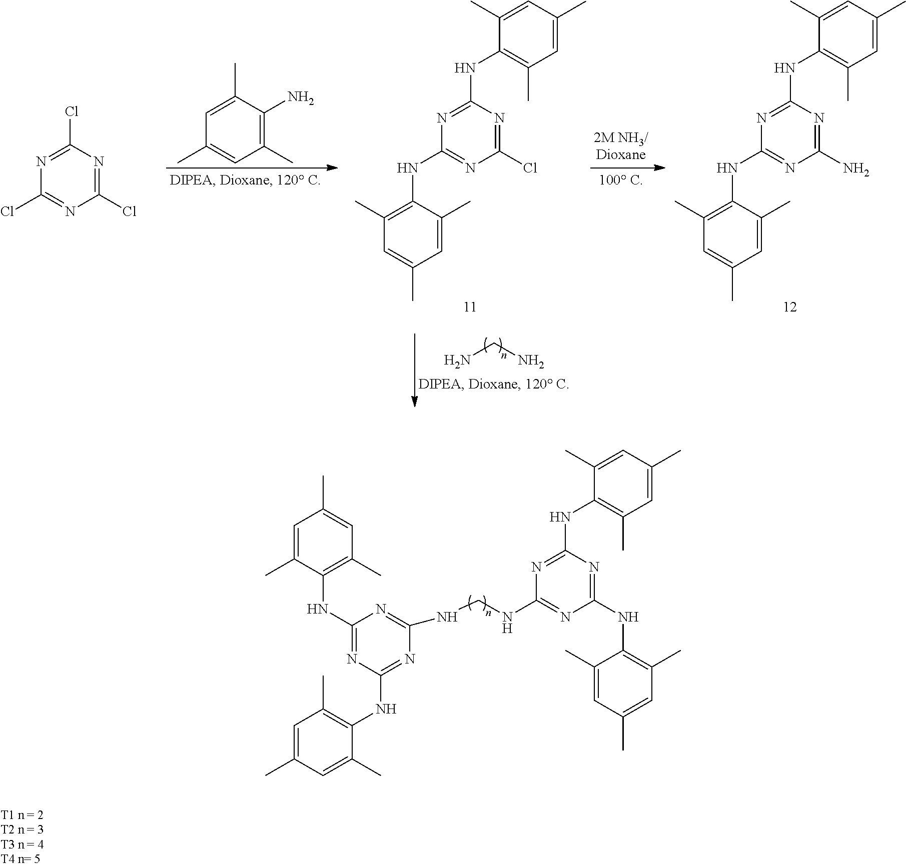

### 6-chloro-N₂,N₄-dimesityl-1,3,5-triazine-2,4-diamine (I1)

To a solution of 2,4,6-trichloro-1,3,5-triazine (0.92 g, 5 mmol) in dioxane (30 mL) was added 2,4,6-trimethylaniline (1.4 mL, 10 mmol) and DIPEA (1.72 mL, 10 mmol) and allowed to reflux for 48 h. Removal of solvent and precipitation with 20% EtOAc in hexanes afforded white solid (1.2 g, 63%); ¹H NMR (DMSO-d₆, 400 MHz) δ 9.20 (s, 1H), 9.07 (s, 1H), 6.91 (s, 2H), 6.85 (s, 2H), 2.21 (s, 6H), 2.13 (s, 6H), 2.03 (s, 6H); MS (ESI) m/z 382 (M+H)⁺; LC-MS (214 nm) tᵣ 19.8 min, 100

### N₂,N₄-dimesityl-1,3,5-triazine-2,4,6-triamine (I2)

I1 (0.38 g, 1 mmol) was dissolved in 2M NH₃/dioxane (10 mL) in a pressure tube and allowed to stir at 100° C. overnight. Removal of solvent and purification by column chromatography using 60% EtOAc in hexanes afforded white solid (0.15 g, 41%); ¹H NMR (DMSO-d₆, 400 MHz) δ 7.84 (br s, 2H), 6.85 (br s, 4H), 6.20 (br s, 2H), 2.20-1.98 (m, 18H); MS (ESI) m/z 363 (M+H)⁺; LC-MS (214 nm) tᵣ 14.7 min, 100%

### N₂,N₂-(ethane-1,2-diyl)bis(N₄,N₆-dimesityl-1,3,5-triazine-2,4,6-triamine) (T1)

To a solution of I1 (0.76 g, 2 mmol) in dioxane (10 mL) was added DIPEA (0.33 mL, 2 mmol) and ethane-1,2-diamine (0.07 mL, 1 mmol) and allowed to reflux for 48 h. Removal of solvent and purification by column chromatography using 70% EtOAc in hexanes afforded white solid (0.29 g, 39%); ¹H NMR (MeOD, 400 MHz) δ 6.92-6.81 (m, 8H), 3.51-3.35 (m, 4H), 2.26-2.00 (m, 36H); MS (ESI) m/z 752 (M+H)⁺; LC-MS (214 nm) tᵣ 18.8 min, 100%

### N₂,N₂-(propane-1,3-diyl)bis(N₄,N₆-dimesityl-1,3,5-triazine-2,4,6-triamine) (T2)

The above compound was prepared from propane-1,3-diamine and I1 using the procedure similar to T1

Yield: 35%

¹H NMR (MeOD, 400 MHz) δ 6.92-6.70 (m, 8H), 3.35 (br s, 4H), 2.28-2.07 (m, 36H), 1.65 (br s, 2H); MS (ESI) m/z 766 (M+H)⁺; LC-MS (214 nm) tᵣ 18.5 min, 100

### N₂,N₂-(butane-1,4-diyl)bis(N₄,N₆-dimesityl-1,3,5-triazine-2,4,6-triamine) (T3)

The above compound was prepared from butane-1,4-diamine and I1 using the procedure similar to T1

Yield: 36%

¹H NMR (DMSO-d₆, 400 MHz) δ 8.20-7.82 (m, 4H), 6.88-6.82 (m, 8H), 6.64 (br s, 2H), 3.31-3.21 (m, 4H), 2.23-2.03 (m, 36H), 1.44 (br s, 4H); MS (ESI) m/z 780 (M+H)⁺; LC-MS (214 nm) tᵣ 18.2 min, 100%

### N₂,N₂-(pentane-1,5-diyl)bis(N₄,N₆-dimesityl-1,3,5-triazine-2,4,6-triamine) (T4)

The above compound was prepared from pentane-1,5-diamine and I1 using the procedure similar to T1

Yield: 30%

¹H NMR (DMSO-d₆, 400 MHz) δ 7.98-7.82 (m, 4H), 6.88-6.83 (m, 8H), 6.57 (br s, 2H), 3.31-3.18 (m, 4H), 2.24-2.07 (m, 36H) 1.44 (br s, 6H); MS (ESI) m/z 794 (M+H)⁺; LC-MS (214 nm) tᵣ 18.7 min, 100%

### Example 3

### Synthesis of Intermediates I3-I5 and Target Compounds T5-T12

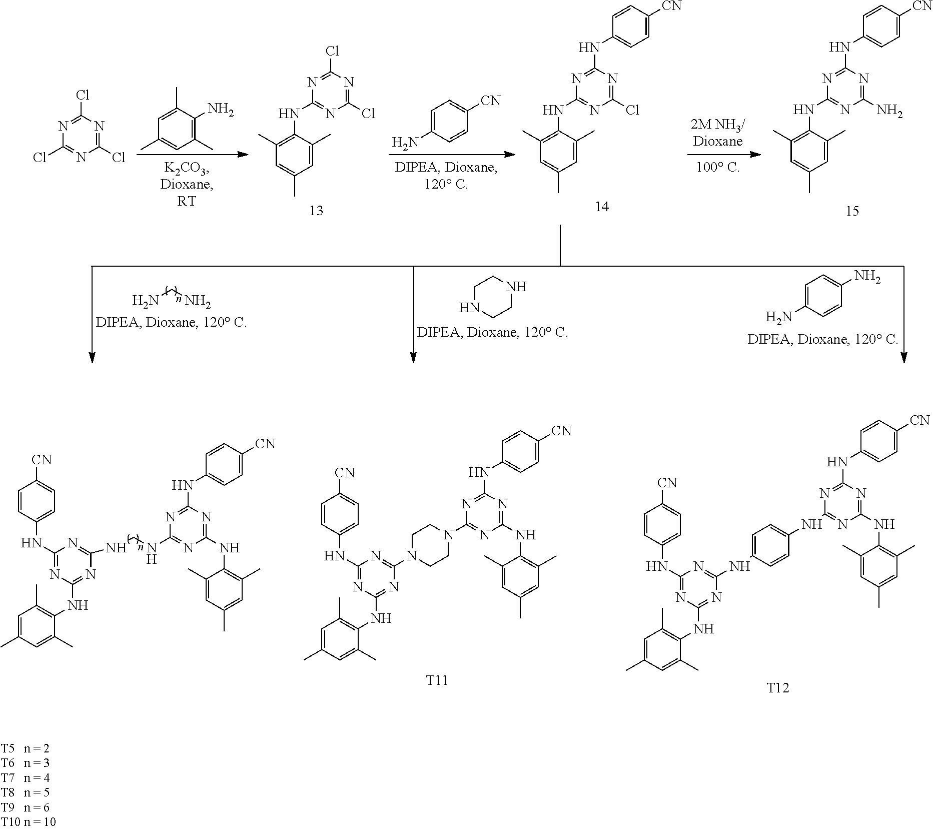

### 4,6-dichloro-N-mesityl-1,3,5-triazin-2-amine (I3)

To a homogenous solution of 2,4,6-trichloro-1,3,5-triazine (3.69 g, 20 mmol) in dioxane (60 mL) was added K₂CO₃ (2.90 g, 21 mmol) and 2,4,6-trimethylaniline (2.96 mL, 21 mmol) and allowed to stir at room temperature for 36 h. Solvents were evaporated and water was added, extracted with EtOAc (3×100 mL), organic layers were washed with NaHCO₃, brine and water, dried and evaporated gave light yellow solid (5.4 g, 95%)

¹H NMR (DMSO-d₆, 400 MHz) δ 10.4 (s, 1H), 6.94 (s, 2H), 2.25 (s, 3H), 2.10 (s, 6H); MS (ESI) m/z 283 (M+H)⁺

### 4-((4-chloro-6-(mesitylamino)-1,3,5-triazin-2-yl)amino)benzonitrile (I4)

To a solution of I3 (0.99 g, 3.5 mmol) in dioxane (30 mL) was added DIPEA (0.64 mL, 3.85 mmol) and 4-aminobenzonitrile (0.41 g, 3.5 mmol) and allowed to stir at 120° C. over weekend. Concentration of the reaction mixture and extraction with EtOAc followed by brine washing afforded dark brown solid. Recrystallisation from EtOAc/hexanes yielded white solid (0.8 g, 63%); ¹H NMR (DMSO-d₆, 400 MHz) δ 10.5 (br s, 1H), 9.7 (br s, 1H), 8.01 (d, 1H, J=9.0 Hz), 7.74 (d, 1H, J=9.0 Hz), 7.51 (br s, 2H), 7.03 (s, 1H), 6.94 (d, 1H, J=9.0 Hz), 2.25 (s, 3H), 2.10 (s, 6H); MS (ESI) m/z 365 (M+H)⁺

### 4-((4-amino-6-(mesitylamino)-1,3,5-triazin-2-yl)amino)benzonitrile (I5)

I4 (0.6 g, 1.65 mmol) was dissolved in 2M NH₃/dioxane (10 mL) in a pressure tube and allowed to stir at 100° C. overnight. Removal of solvent and purification by column chromatography using 60 EtOAc in hexanes afforded white solid (0.5 g, 88%); ¹H NMR (DMSO-d₆, 400 MHz) δ 9.5 (br s, 1H), 8.28 (br s, 1H), 8.01 (d, 1H, J=8.6 Hz), 7.77 (br s, 1H), 7.64 (d, 1H, J=8.6 Hz), 7.45 (s, 1H), 6.85 (s, 2H), 6.57 (s, 2H), 2.25 (s, 3H), 2.10 (s, 6H); MS (ESI) m/z 346 (M+H)⁺; LC-MS (214 nm) tᵣ 14.5 min, 95%

### 4,4′-((6,6′-(ethane-1,2-diylbis(azanediyl))bis(4-(mesitylamino)-1,3,5-triazine-6,2-diyl))bis(azanediyl))dibenzonitrile (T5)

The above compound was prepared from ethane-1,2-diamine and I4 using the procedure similar to T1

Yield: 21%

¹H NMR (MeOD, 400 MHz) δ 8.09 (br s, 2H), 7.75 (br s, 4H), 7.48 (br s, 2H), 7.12 (br s, 4H), 3.86-3.51 (m, 4H), 2.50 (br s, 6H), 2.32 (br s, 12H); MS (ESI) m/z 718 (M+H)⁺; LC-MS (214 nm) tᵣ 20.7 min, 100%

### 4,4′-((6,6′-(propane-1,3-diylbis(azanediyl))bis(4-(mesitylamino)-1,3,5-triazine-6,2-diyl))bis(azanediyl))dibenzonitrile (T6)

The above compound was prepared from propane-1,3-diamine and I4 using the procedure similar to T1

Yield: 37%

¹H NMR (MeOD, 400 MHz) δ 7.97 (br s, 2H), 7.61 (br s, 4H), 7.36 (br s, 2H), 6.96 (br s, 4H), 3.56-3.49 (m, 4H), 2.33 (br s, 6H), 2.19 (br s, 12H), 1.88 (br s, 2H); MS (ESI) m/z 732 (M+H)⁺; LC-MS (214 nm) tᵣ 21.9 min, 100%

### 4,4′-((6,6′-(butane-1,4-diylbis(azanediyl))bis(4-(mesitylamino)-1,3,5-triazine-6,2-diyl))bis(azanediyl))dibenzonitrile (T7)

The above compound was prepared from butane-1,4-diamine and I4 using the procedure similar to T1

Yield: 51%

¹H NMR (MeOD, 400 MHz) δ 7.96 (br s, 2H), 7.61 (br s, 4H), 7.36 (br s, 2H), 6.90 (br s, 4H), 3.56-3.49 (m, 4H), 2.33 (br s, 6H), 2.19 (br s, 12H), 1.76-1.66 (m, 4H); MS (ESI) m/z 746 (M+H)⁺; LC-MS (214 nm) tᵣ 20.7 min, 100%

### 4,4′-((6,6′-(pentane-1,5-diylbis(azanediyl))bis(4-(mesitylamino)-1,3,5-triazine-6,2-diyl))bis(azanediyl))dibenzonitrile (T8)

The above compound was prepared from pentane-1,5-diamine and I4 using the procedure similar to T1

Yield: 26%

¹H NMR (MeOD, 400 MHz) δ 7.98 (br s, 2H), 7.61 (br s, 4H), 7.37 (br s, 2H), 6.93 (br s, 4H), 3.42-3.37 (m, 4H), 2.33 (br s, 6H), 2.19 (br s, 12H), 1.72-1.66 (m, 4H) 1.41 (br s, 2H); MS (ESI) m/z 760 (M+H)⁺; LC-MS (214 nm) tᵣ 20.5 min, 100%

### 4,4′-((6,6′-(hexane-1,6-diylbis(azanediyl))bis(4-(mesitylamino)-1,3,5-triazine-6,2-diyl))bis(azanediyl))dibenzonitrile (T9)

The above compound was prepared from hexane-1,6-diamine and I4 using the procedure similar to T1

Yield: 25%

¹H NMR (MeOD, 400 MHz) δ 7.98 (br s, 2H), 7.61 (br s, 4H), 7.37 (d, J=7.6 Hz, 2H), 6.92 (br s, 4H), 3.42 (br s, 4H), 2.33 (br s, 6H), 2.19 (br s, 12H), 1.66 (br s, 4H) 1.47 (br s, 4H); MS (ESI) m/z 774 (M+H)⁺; LC-MS (214 nm) tᵣ 20.6 min, 100%

### 4,4′-((6,6′-(decane-1,10-diylbis(azanediyl))bis(4-(mesitylamino)-1,3,5-triazine-6,2-diyl))bis(azanediyl))dibenzonitrile (T10)

The above compound was prepared from decane-1,10-diamine and I4 using the procedure similar to T1

Yield: 15%

¹H NMR (MeOD, 400 MHz) δ 7.97 (br s, 2H), 7.66-7.58 (m, 4H), 7.37 (d, J=8.0 Hz, 2H), 6.91 (br s, 4H), 3.40 (br s, 4H), 2.31 (br s, 6H), 2.19 (br s, 12H), 1.6 (br s, 4H), 1.35 (br s, 12H); MS (ESI) m/z 830 (M+H)⁺; LC-MS (214 nm) tᵣ 23.4 min, 100%

### 4,4′-((6,6′-(piperazine-1,4-diyl)bis(4-(mesitylamino)-1,3,5-triazine-6,2-diyl))bis(azanediyl))dibenzonitrile (T11)

The above compound was prepared from piperazine and I4 using the procedure similar to T1 Yield: 14%

¹H NMR (DMSO-d₆, 400 MHz) δ 9.56 (br s, 2H), 8.52 (br s, 2H), 8.0 (br s, 1H), 7.72 (br s, 4H), 7.49 (d, J=7.0 Hz, 3H), 6.92 (br s, 4H), 3.9 (br s, 8H), 2.23 (br s, 6H), 2.13 (br s, 12H); MS (ESI) m/z 744 (M+H)⁺; LC-MS (214 nm) tᵣ 23.2 min, 98%

### 4,4′-((6,6′-(1,4-phenylenebis(azanediyl))bis(4-(mesitylamino)-1,3,5-triazine-6,2-diyl))bis(azanediyl))dibenzonitrile (T12)

The above compound was prepared from benzene-1,4-diamine and I4 using the procedure similar to T1

Yield: 10%

¹H NMR (DMSO-d₆, 400 MHz) δ 9.62 (br s, 1H), 9.51 (br s, 1H), 9.14 (br s, 2H), 8.55 (br s, 2H), 8.07 (br s, 2H), 7.84 (br s, 2H), 7.68 (br s, 4H), 7.49 (br s, 4H), 6.92 (br s, 4H), 2.26 (br s, 6H), 2.13 (br s, 12H); MS (ESI) m/z 766 (M+H)⁺; LC-MS (214 nm) tᵣ 22.6 min, 96%

### Example 4

### Synthesis of Intermediates I6-I8 and Target Compounds T13-T15

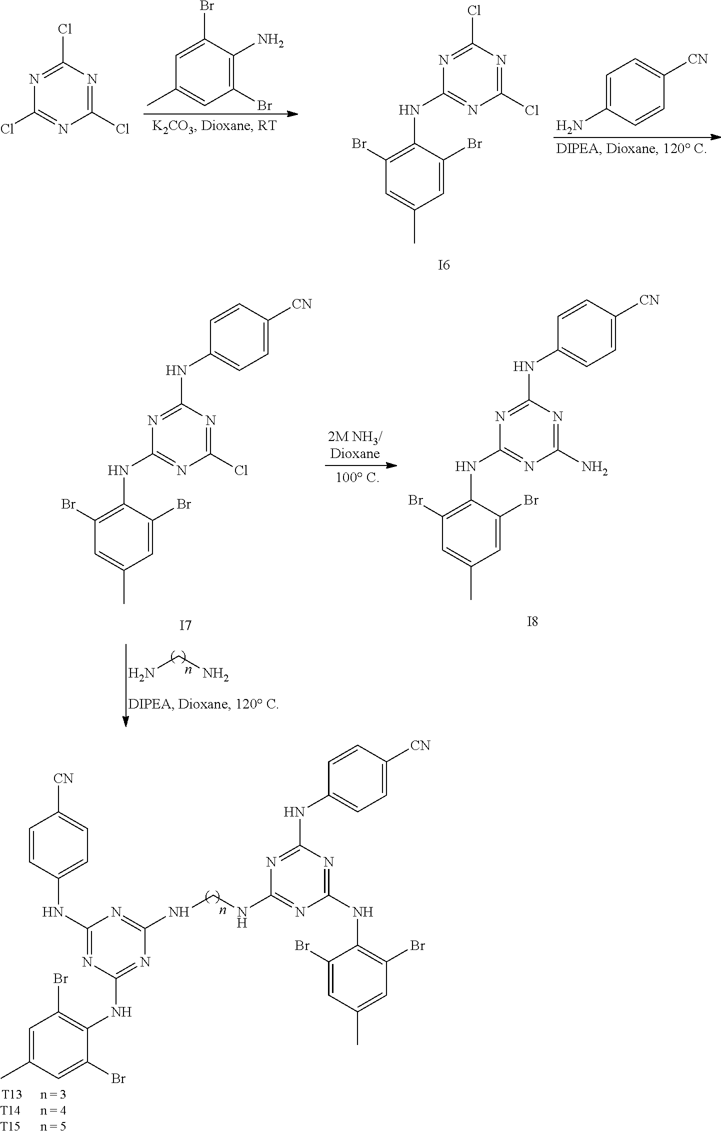

### 4,6-dichloro-N-(2,6-dibromo-4-methylphenyl)-1,3,5-triazin-2-amine (I6)

The above intermediate was prepared from 2,4,6-trichloro-1,3,5-triazine and 2,6-dibromo-4-methylaniline using the procedure similar to I3; Yield 74%; MS (ESI) m/z 414 (M+H)⁺

### 4-((4-chloro-6-((2,6-dibromo-4-methylphenyl)amino)-1,3,5-triazin-2-yl)amino)benzonitrile (I7)

The above intermediate was prepared from I6 using the procedure similar to I4.

Yield: 65%

¹H NMR (DMSO-d₆, 400 MHz) δ 10.65 (br s, 1H), 10.20 (br s, 1H), 8.0 (d, 1H, J=8.7 Hz), 7.80 (d, 1H, J=8.7 Hz), 7.66 (br s, 1H), 7.59 (s, 1H), 7.53 (br s, 2H), 2.38 (br s, 3H); MS (ESI) m/z 497 (M+H)⁺; HPLC (214 nm) tᵣ 26.24 min, 100%;

### 4-((4-chloro-6-((2,6-dibromo-4-methylphenyl)amino)-1,3,5-triazin-2-yl)amino)benzonitrile (I8)

The above intermediate was prepared from I7 using the procedure similar to I5.

Yield: 51%

¹H NMR (MeOD, 400 MHz) δ 7.96 (d, 1H, J=8.7 Hz), 7.62 (d, 2H, J=8.5 Hz), 7.55 (s, 2H), 7.40 (br s, 1H), 2.38 (br s, 3H); MS (ESI) m/z 476 (M+H)⁺; HPLC (214 nm) tᵣ 19.6 min, 100%

### 4,4′-((6,6′-(propane-1,3-diylbis(azanediyl))bis(4-((2,6-dibromo-4-methylphenyl)amino)-1,3,5-triazine-6,2-diyl))bis(azanediyl))dibenzonitrile (T13)

The above compound was prepared from propane-1,3-diamine and I7 using the procedure similar to T1

Yield: 61%

¹H NMR (MeOD, 400 MHz) δ 8.29-7.4 (br s, 12H), 3.6 (br s, 4H), 2.4 (br s, 6H), 1.85 (br s, 2H); MS (ESI) m/z 991(M+H)⁺; LC-MS (214 nm) tᵣ 22.2 min, 100%

### 4,4′-((6,6′-(butane-1,4-diylbis(azanediyl))bis(4-((2,6-dibromo-4-methylphenyl)amino)-1,3,5-triazine-6,2-diyl))bis(azanediyl))dibenzonitrile (T14)

The above compound was prepared from butane-1,4-diamine and I7 using the procedure similar to T1

Yield: 43%

¹H NMR (MeOD, 400 MHz) δ 7.9-7.5 (br s, 12H), 3.44 (br s, 4H), 2.34 (br s, 6H), 1.67 (br s, 4H); MS (ESI) m/z 1005 (M+H)⁺; HPLC (214 nm) tᵣ 28.2 min, 100%

### 4,4′-((6,6′-(pentane-1,5-diylbis(azanediyl))bis(4-((2,6-dibromo-4-methylphenyl)amino)-1,3,5-triazine-6,2-diyl))bis(azanediyl))dibenzonitrile (T15)

The above compound was prepared from pentane-1,5-diamine and I7 using the procedure similar to T1

Yield: 52%

¹H NMR (MeOD, 400 MHz) δ 7.7-7.1 (br s, 12H), 3.11 (br s, 4H), 2.1 (br s, 6H), 1.7 (br s, 4H), 1.41 (br s, 2H); MS (ESI) m/z 1019 (M+H)⁺; HPLC (214 nm) tᵣ 28.8 min, 100%

### Example 5

### Synthesis of Intermediates I9-I11 and Target Compounds T16-T18

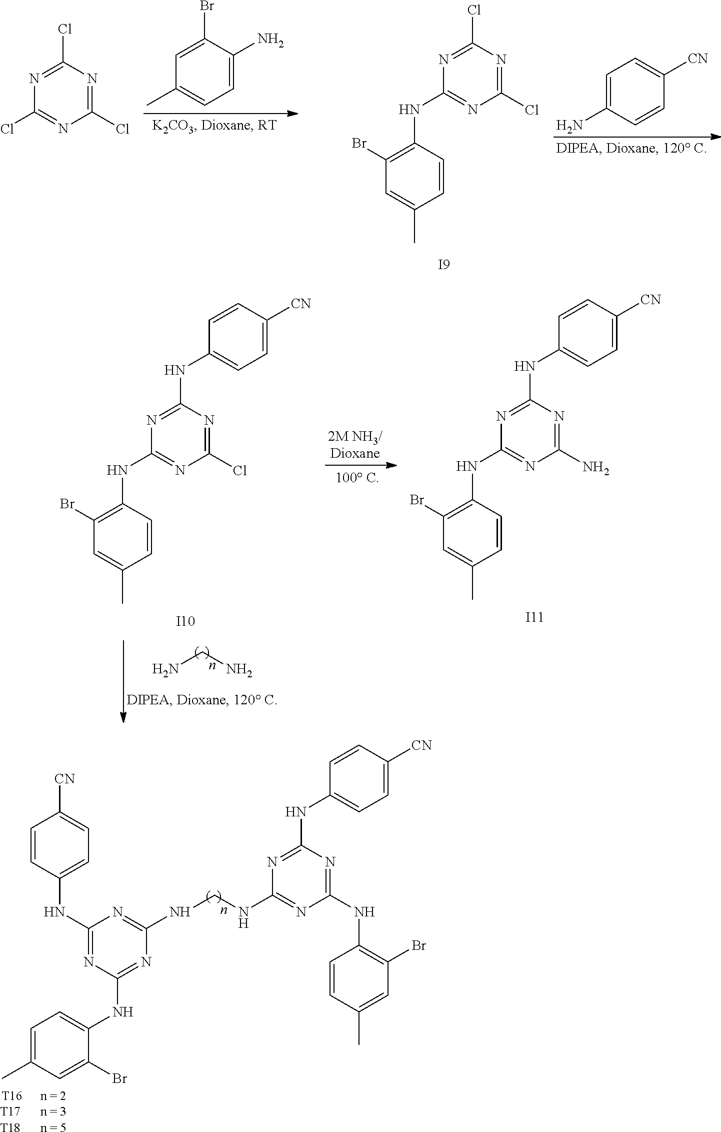

### N-(2-bromo-4-methylphenyl)-4,6-dichloro-1,3,5-triazin-2-amine (I9)

The above intermediate was prepared from 2,4,5-trichloro-1,3,5-triazine and 2-bromo-4-methylaniline using the procedure similar to I3; Yield 87%; MS (ESI) m/z 335 (M+H)⁺

### 4-((4-((2-bromo-4-methylphenyl)amino)-6-chloro-1,3,5-triazin-2-yl)amino)benzonitrile (I10)

The above intermediate was prepared from I9 using the procedure similar to I4.

Yield: 77%

¹H NMR (DMSO-d₆, 400 MHz) δ 10.67 (br s, 1H), 10.20 (br s, 1H), 7.57 (br s, 5H), 7.35 (br s, 1H), 7.26 (br s, 1H), 2.36 (br s, 3H); MS (ESI) m/z 417 (M+H)⁺; HPLC (214 nm) tᵣ 27.5 min, 98%

### 4-((4-amino-6-((2-bromo-4-methylphenyl)amino)-1,3,5-triazin-2-yl)amino)benzonitrile (I11)

The above intermediate was prepared from I10 using the procedure similar to I5.

Yield: 14%

¹H NMR (DMSO-d₆, 400 MHz) δ 7.86 (br s, 3H), 7.55 (d, 2H, J=8.0 Hz), 7.47 (br s, 1H), 7.18 (d, 1H, J=7.4 Hz), 2.35 (br s, 3H); MS (ESI) m/z 397 (M+H)⁺; HPLC (214 nm) tᵣ 18.8 min, 95%

### 4,4′-((6,6′-(ethane-1,2-diylbis(azanediyl))bis(4-((2-bromo-4-methylphenyl)amino)-1,3,5-triazine-6,2-diyl))bis(azanediyl))dibenzonitrile (T16)

The above compound was prepared from ethane-1,2-diamine and I10 using the procedure similar to T1

Yield: 37%

¹H NMR (MeOD, 400 MHz) δ 7.77 (br s, 6H), 7.40 (br s, 6H), 7.04 (br s, 2H), 3.61 (br s, 4H), 2.27 (br s, 6H); MS (ESI) m/z 819 (M+H)⁺; LC-MS (214 nm) tᵣ 23.4 min, 100

### 4,4′-((6,6′-(propane-1,3-diylbis(azanediyl))bis(4-((2-bromo-4-methylphenyl)amino)-1,3,5-triazine-6,2-diyl))bis(azanediyl))dibenzonitrile (T17)

The above compound was prepared from propane-1,3-diamine and I10 using the procedure similar to T1

Yield: 20%

¹H NMR (DMSO-d₆, 400 MHz) δ 9.62 (s, 2H), 8.31 (br s, 2H), 7.94 (br s, 4H), 7.40 (br s, 6H), 7.27 (br s, 2H), 7.16 (br s, 4H), 3.39 (br s, 4H), 2.27 (br s, 6H), 1.90 (br s, 2H); MS (ESI) m/z 833 (M+H)⁺; LC-MS (214 nm) tᵣ 23.7 min, 100%

### 4,4′-((6,6′-(pentane-1,5-diylbis(azanediyl))bis(4-((2-bromo-4-methylphenyl)amino)-1,3,5-triazine-6,2-diyl))bis(azanediyl))dibenzonitrile (T18)

The above compound was prepared from pentane-1,5-diamine and I10 using the procedure similar to T1

Yield: 26%

¹H NMR (MeOD, 400 MHz) δ 7.81 (br s, 6H), 7.33 (br s, 6H), 7.04 (br s, 2H), 3.35 (br s, 4H), 2.30 (br s, 6H), 1.7 (br s, 4H), 1.44 (br s, 2H); MS (ESI) m/z 861 (M+H)⁺; LC-MS (214 nm) tᵣ 23.9 min, 100%

### Example 6

### Synthesis of intermediates I12-I17 and target compounds T19-T24

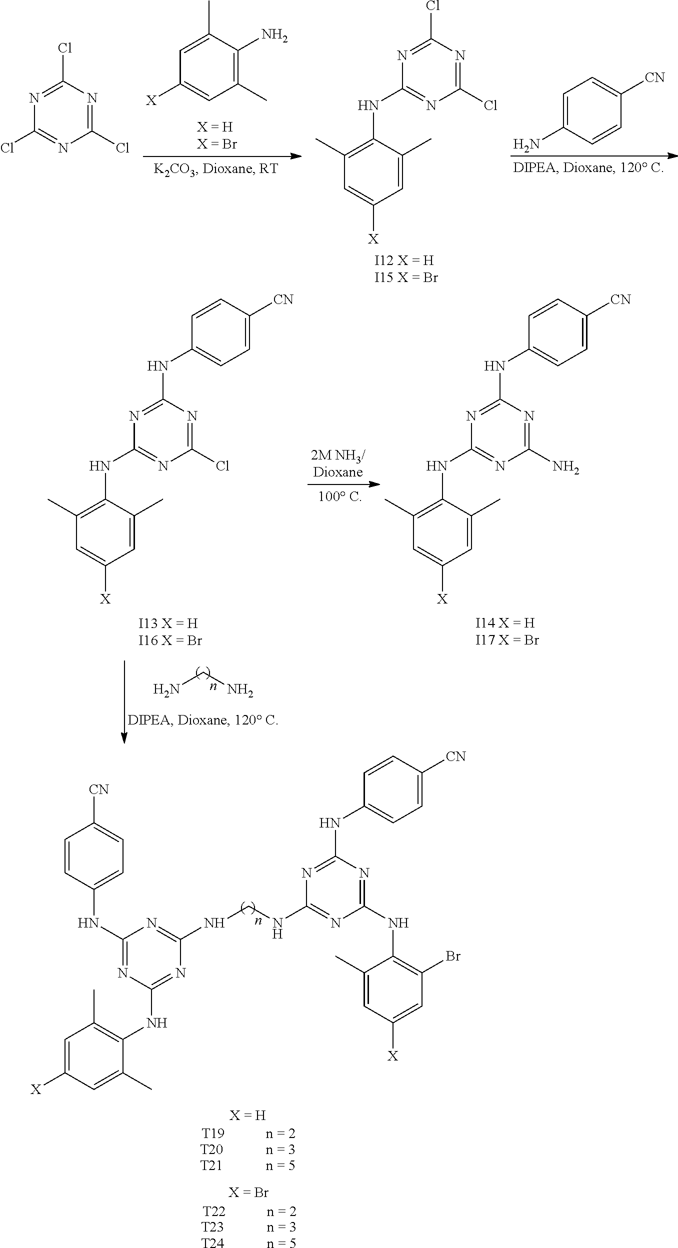

### 4,6-dichloro-N-(2,6-dimethylphenyl)-1,3,5-triazin-2-amine (I12)

The above intermediate was prepared from 2,4,6-trichloro-1,3,5-triazine and 2,6-dimethylaniline using the procedure similar to I3

Yield: 97%

¹H NMR (DMSO-d₆, 400 MHz) δ 10.53 (br s, 1H), 7.13-7.01 (m, 3H), 2.13 (s, 6H); MS (ESI) m/z 270 (M+H)⁺

### 4-((4-chloro-6-((2,6-dimethylphenyl)amino)-1,3,5-triazin-2-yl)amino)benzonitrile (I13)

The above intermediate was prepared from I12 using the procedure similar to I4

Yield: 64%

¹H NMR (MeOD, 400 MHz) δ 8.00 (br s, 1H), 7.78 (br s, 1H), 7.49 (br s, 1H), 7.18-7.08 (m, 4H), 2.16 (s, 6H); MS (ESI) m/z 351 (M+H)⁺; LC-MS (214 nm) tᵣ 18.0 min, 100%

### 4-((4-amino-6-((2,6-dimethylphenyl)amino)-1,3,5-triazin-2-yl)amino)benzonitrile (I14)

The above intermediate was prepared from I13 using the procedure similar to I5

Yield: 78%

¹H NMR (MeOD, 400 MHz) δ 8.02 (br s, 1H), 7.62 (br s, 2H), 7.31 (br s, 1H), 7.15 (br s, 3H), 2.26 (br s, 6H); MS (ESI) m/z 332 (M+H)⁺; LC-MS (214 nm) tᵣ 14.7 min, 100%

### 4,4′-((6,6′-(ethane-1,2-diylbis(azanediyl))bis(4-((2,6-dimethylphenyl)amino)-1,3,5-triazine-6,2-diyl))bis(azanediyl))dibenzonitrile (T19)

The above compound was prepared from ethane-1,2-diamine and I13 using the procedure similar to T1

Yield: 23%

¹H NMR (MeOD, 400 MHz) δ 7.90 (br s, 1H), 7.55 (br s, 4H), 7.28 (br s, 3H), 7.10 (br s, 6H), 3.69-3.54 (m, 4H), 2.17 (br s, 12H); MS (ESI) m/z 690 (M+H)⁺; LC-MS (214 nm) tᵣ 19.3 min, 100%

### 4,4′-((6,6′-(propane-1,3-diylbis(azanediyl))bis(4-((2,6-dimethylphenyl)amino)-1,3,5-triazine-6,2-diyl))bis(azanediyl))dibenzonitrile (T20)

The above compound was prepared from propane-1,3-diamine and I13 using the procedure similar to T1

Yield: 37%

¹H NMR (MeOD, 400 MHz) δ 7.90 (br s, 1H), 7.60 (br s, 4H), 7.25 (br s, 3H), 7.12 (br s, 6H), 3.69-3.56 (m, 4H), 2.22 (br s, 12H), 1.90-1.75 (m, 2H); MS (ESI) m/z 704 (M+H)⁺; LC-MS (214 nm) tᵣ 20.4 min, 100

### 4,4′-((6,6′-(pentane-1,5-diylbis(azanediyl))bis(4-((2,6-dimethylphenyl)amino)-1,3,5-triazine-6,2-diyl))bis(azanediyl))dibenzonitrile (T21)

The above compound was prepared from pentane-1,5-diamine and I13 using the procedure similar to T1

Yield: 33%

¹H NMR (MeOD, 400 MHz) δ 7.90 (br s, 1H), 7.60 (br s, 4H), 7.35 (br s, 3H), 7.11 (br s, 6H), 3.6-3.5 (m, 4H), 2.26 (br s, 12H), 1.75-1.60 (m, 4H), 1.5-1.4 (m, 2H); MS (ESI) m/z 732 (M+H)⁺; LC-MS (214 nm) tᵣ 18.9 min, 100%

### N-(4-bromo-2,6-dimethylphenyl)-4,6-dichloro-1,3,5-triazin-2-amine (I15)

The above intermediate was prepared from 2,4,6-trichloro-1,3,5-triazine and 4-bromo-2,6-dimethylaniline using the procedure similar to I3

Yield: 86%

¹H NMR (DMSO-d₆, 400 MHz) δ 10.54 (s, 1H), 7.36 (s, 2H), 2.06 (s, 6H); MS (ESI) m/z 349 (M+H)⁺

### 4-((4-((4-bromo-2,6-dimethylphenyl)amino)-6-chloro-1,3,5-triazin-2-yl)amino)benzonitrile (I16)

The above intermediate was prepared from I15 using the procedure similar to I4

Yield: 65%

¹H NMR (MeOD, 400 MHz) δ 7.72-7.28 (m, 6H), 2.21 (s, 6H); MS (ESI) m/z 431 (M+H)⁺; LC-MS (214 nm) tᵣ 19.2 min, 100%

### 4-((4-amino-6-((4-bromo-2,6-dimethylphenyl)amino)-1,3,5-triazin-2-yl)amino)benzonitrile (I17)

The above intermediate was prepared from I16 using the procedure similar to I5

Yield: 56%

¹H NMR (MeOD, 400 MHz) δ 8.00 (br s, 1H), 7.63 (br s, 2H), 7.42-7.34 (m, 3H), 2.24 (s, 6H); MS (ESI) m/z 411 (M+H)⁺; LC-MS (214 nm) tᵣ 17.0 min, 100%

### 4,4′-((6,6′-(ethane-1,2-diylbis(azanediyl))bis(4-((4-bromo-2,6-dimethylphenyl)amino)-1,3,5-triazine-6,2-diyl))bis(azanediyl))dibenzonitrile (T22)

The above compound was prepared from ethane-1,2-diamine and I16 using the procedure similar to T1

Yield: 41%

¹H NMR (MeOD, 400 MHz) δ 7.96 (br s, 2H), 7.63 (br s, 4H), 7.33 (br s, 6H), 3.65 (br s, 4H), 2.10 (br s, 12H); MS (ESI) m/z 848 (M+H)⁺; LC-MS (214 nm) tᵣ 21.7 min, 100%

### 4,4′-((6,6′-(propane-1,3-diylbis(azanediyl))bis(4-((4-bromo-2,6-dimethylphenyl)amino)-1,3,5-triazine-6,2-diyl))bis(azanediyl))dibenzonitrile (T23)

The above compound was prepared from propane-1,3-diamine and I16 using the procedure similar to T1

Yield: 37%

¹H NMR (MeOD, 400 MHz) δ 8.00 (br s, 2H), 7.64 (br s, 4H), 7.40 (br s, 6H), 3.69 (br s, 4H), 2.10 (br s, 12H), 1.88 (br s, 2H); MS (ESI) m/z 862 (M+H)⁺; LC-MS (214 nm) tᵣ 22.3 min, 100%.

### 4,4′-((6,6′-(pentane-1,5-diylbis(azanediyl))bis(4-((4-bromo-2,6-dimethylphenyl)amino)-1,3,5-triazine-6,2-diyl))bis(azanediyl))dibenzonitrile (T24)

The above compound was prepared from pentane-1,5-diamine and I16 using the procedure similar to T1

Yield: 42%

¹H NMR (MeOD, 400 MHz) δ 7.98 (br s, 2H), 7.62 (br s, 4H), 7.31 (br s, 6H), 3.34 (br s, 4H), 2.10 (br s, 12H), 1.81 (br s, 4H), 1.56 (br s, 2H); MS (ESI) m/z 890 (M+H)⁺; LC-MS (214 nm) tᵣ 21.6 min, 100%

### Example 7

### Synthesis of Target Compounds T25 and T26

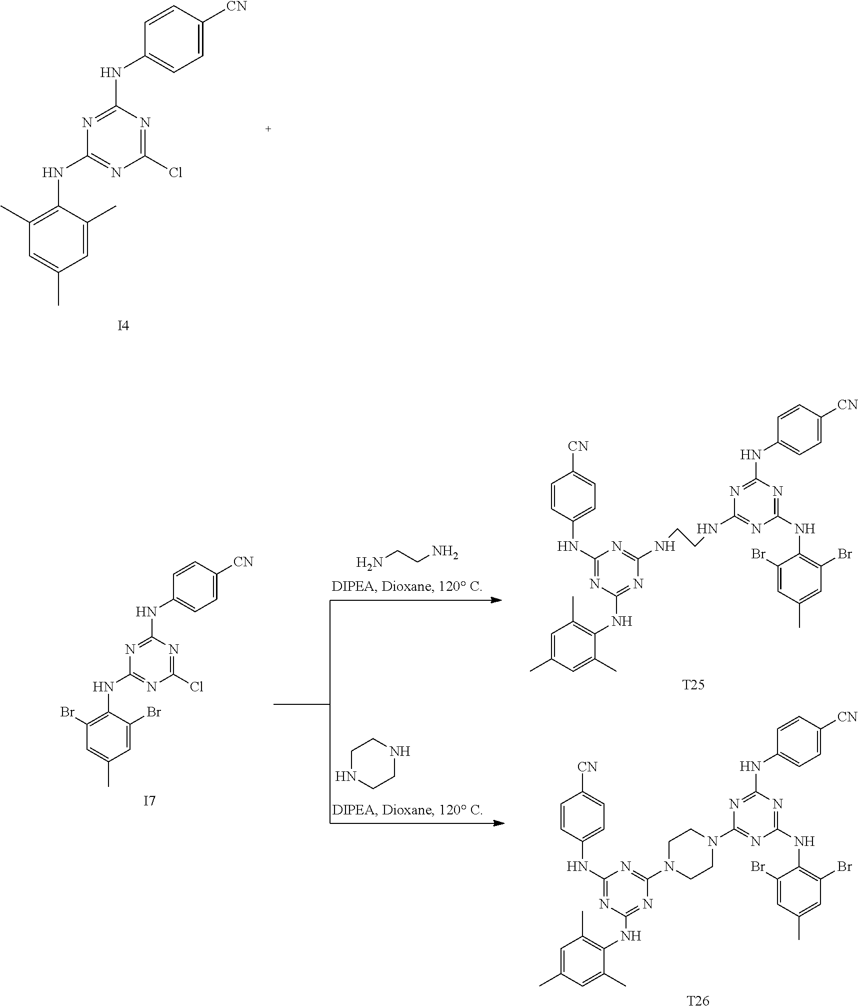

### 4-((4-((2-((4-((4-cyanophenyl)amino)-6-((2,6-dibromo-4-methylphenyl)amino)-1,3,5-triazin-2-yl)amino)ethyl)amino)-6-(mesitylamino)-1,3,5-triazin-2-yl)amino)benzonitrile (T25)

To a solution of I7 (0.24 g, 0.5 mmol) in dioxane (10 mL) was added DIPEA (0.09 mL, 0.53 mmol) and ethane-1,2-diamine (0.032 mL, 0.5 mmol) and allowed to reflux for 12 h and then I4 (0.2 g, 0.5 mmol) was added and subsequently refluxd for 24 h. Removal of solvent and purification by column chromatography using 70% EtOAc in hexanes afforded white solid (0.05 g, 11%). ¹H NMR (MeOD, 400 MHz) δ 7.91 (br s, 2H), 7.57 (br s, 4H), 7.31 (br s, 2H), 6.93 (br s, 4H), 3.67-3.58 (m, 4H), 2.33 (br s, 6H), 2.17 (br s, 6H); MS (ESI) m/z 847 (M+H)⁺; LC-MS (214 nm) tᵣ 20.8 min, 100%

### 4-((4-(4-(4-((4-cyanophenyl)amino)-6-((2,6-dibromo-4-methylphenyl)amino)-1,3,5-triazin-2-yl)piperazin-1-yl)-6-(mesitylamino)-1,3,5-triazin-2-yl)amino)benzonitrile (T26)

The above compound was prepared from intermediates I4, I7 and piperazine using the procedure similar to T25

Yield: 0.07 g, 10%

¹H NMR (DMSO-d₆, 400 MHz) δ 9.64 (br s, 1H), 9.56 (br s, 1H), 9.13 (br s, 1H), 8.57 (br s, 1H), 8.0 (br s, 1H), 7.72 (br s, 4H), 7.49 (br s, 3H), 6.94 (br s, 4H), 3.9 (br s, 8H), 2.23 (br s, 6H), 2.13 (br s, 6H); MS (ESI) m/z 874 (M+H)⁺; LC-MS (214 nm) tᵣ 22.2 min, 100%

### Example 8

### Synthesis of Target Compounds T27 and T28

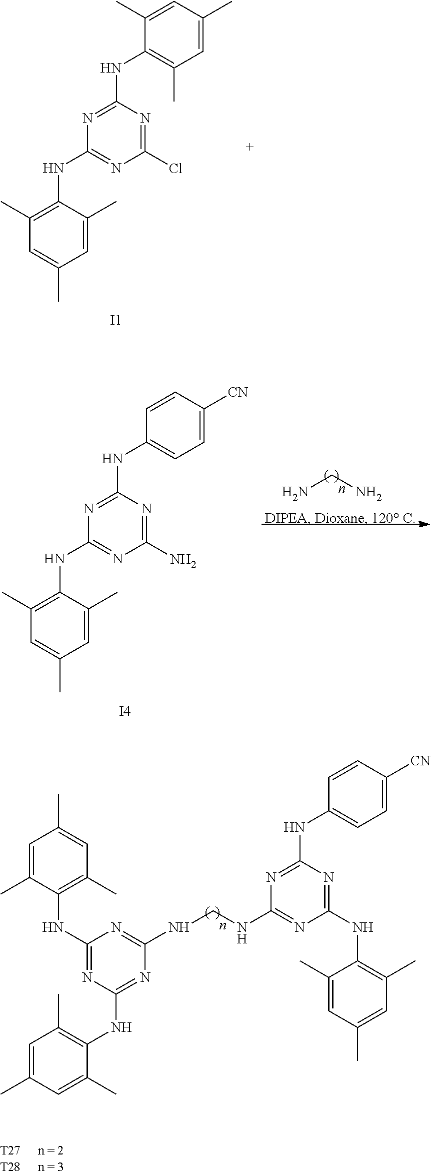

### 4-((4-((2-((4,6-bis(mesitylamino)-1,3,5-triazin-2-yl)amino)ethyl)amino)-6-(mesitylamino)-1,3,5-triazin-2-yl)amino)benzonitrile (T27)

The above compound was prepared from intermediates I1, I4 and ethane-1,2-diamine using the procedure similar to T25

Yield: 34%

¹H NMR (MeOD, 400 MHz) δ 8.48-7.47 (m, 4H), 6.94-6.83 (m, 6H), 3.51-3.46 (m, 4H), 2.26-2.03 (m, 27H); MS (ESI) m/z 735 (M+H)⁺; LC-MS (214 nm) tᵣ 19.4 min, 100

### 4-((4-((3-((4,6-bis(mesitylamino)-1,3,5-triazin-2-yl)amino)propyl)amino)-6-(mesitylamino)-1,3,5-triazin-2-yl)amino)benzonitrile (T28)

The above compound was prepared from intermediates I1, I4 and propane-1,3-diamine using the procedure similar to T25

Yield: 35%

¹H NMR (MeOD, 400 MHz) δ 7.58 (br s, 2H), 7.30 (br s, 2H), 6.94-6.74 (m, 6H), 3.35-3.31 (br s, 4H), 2.32-2.0 (m, 27H), 1.75 (br s, 2H); MS (ESI) m/z 749 (M+H)⁺; LC-MS (214 nm) tᵣ 19.8 min, 100%

### Example 9

### Synthesis of intermediates I18-I20 and target compounds T29 and T30

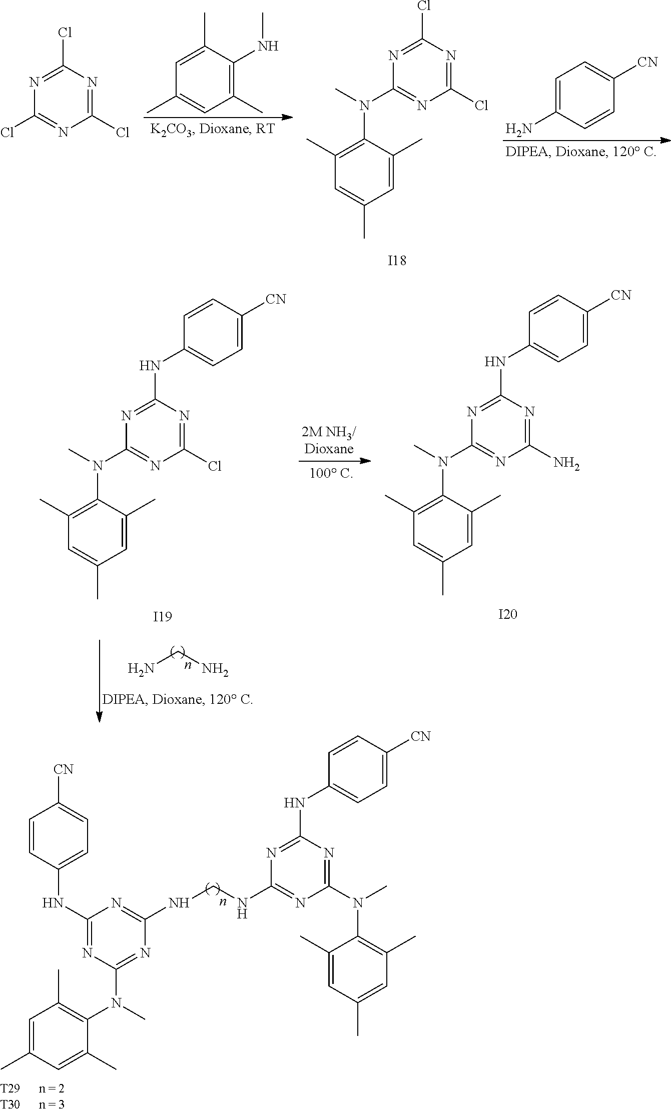

### 4,6-dichloro-N-mesityl-N-methyl-1,3,5-triazin-2-amine (I18)

The above intermediate was prepared from 2,4,6-trichloro-1,3,5-triazine and N,2,4,6-tetramethylaniline using the procedure similar to I3

Yield: 94%

¹H NMR (DMSO-d₆, 400 MHz) δ 6.97 (s, 2H), 2.26 (s, 3H), 2.12 (s, 3H), 2.06 (s, 6H); MS (ESI) m/z 298(M+H)⁺

### 4-((4-chloro-6-(mesityl(methyl)amino)-1,3,5-triazin-2-yl)amino)benzonitrile (I19)

The above intermediate was prepared from I18 using the procedure similar to I4.

Yield: 29%

MS (ESI) m/z 380(M+H)⁺

### 4-((4-amino-6-(mesityl(methyl)amino)-1,3,5-triazin-2-yl)amino)benzonitrile (I20)

The above intermediate was prepared from I19 using the procedure similar to I5.

¹H NMR (MeOD, 400 MHz) δ 7.98 (s, 1H), 7.61 (s, 1H), 7.42 (s, 2H), 7.22 (s, 2H), 7.00 (s, 2H), 2.23 (s, 3H), 2.13 (s, 3H), 2.03 (s, 6H); MS (ESI) m/z 360(M+H)⁺; LC-MS (214 nm) tᵣ 17.6 min, 100%

### 4,4′-((6,6′-(ethane-1,2-diylbis(azanediyl))bis(4-(mesityl(methyl)amino)-1,3,5-triazine-6,2-diyl))bis(azanediyl))dibenzonitrile (T29)

The above compound was prepared from ethane-1,2-diamine and I19 using the procedure similar to T1

Yield: 19%

¹H NMR (MeOD, 400 MHz) δ 7.43 (br s, 4H), 7.22 (br s, 4H), 6.96-6.85 (m, 4H), 3.66-3.48 (m, 4H), 2.38 (s, 6H), 2.00 (br s, 18H); MS (ESI) m/z 746 (M+H)⁺; LC-MS (214 nm) tᵣ 24.1 min, 100%

### 4,4′-((6,6′-(propane-1,3-diylbis(azanediyl))bis(4-(mesityl(methyl)amino)-1,3,5-triazine-6,2-diyl))bis(azanediyl))dibenzonitrile (T30)

The above compound was prepared from propane-1,3-diamine and I19 using the procedure similar to T1.

Yield: 29%

¹H NMR (MeOD, 400 MHz) δ 7.50 (br s, 4H), 7.25 (br s, 4H), 6.97 (br s, 4H), 3.52-3.48 (m, 4H), 2.35 (s, 6H), 2.04 (br s, 18H), 1.80 (br s, 2H); MS (ESI) m/z 760 (M+H)⁺; LC-MS (214 nm) tᵣ 23.8 min, 100%

### Example 10

### Synthesis of Intermediates I21-I27 and Target Compounds T31-T37

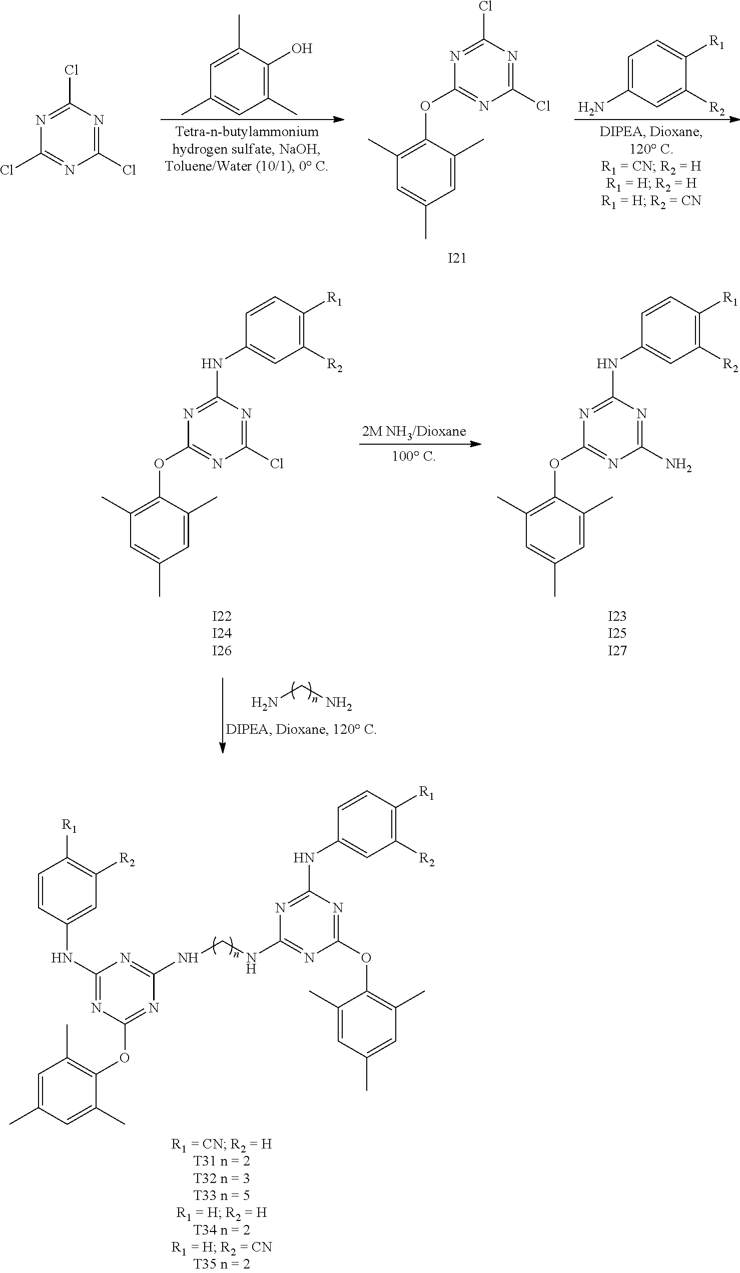

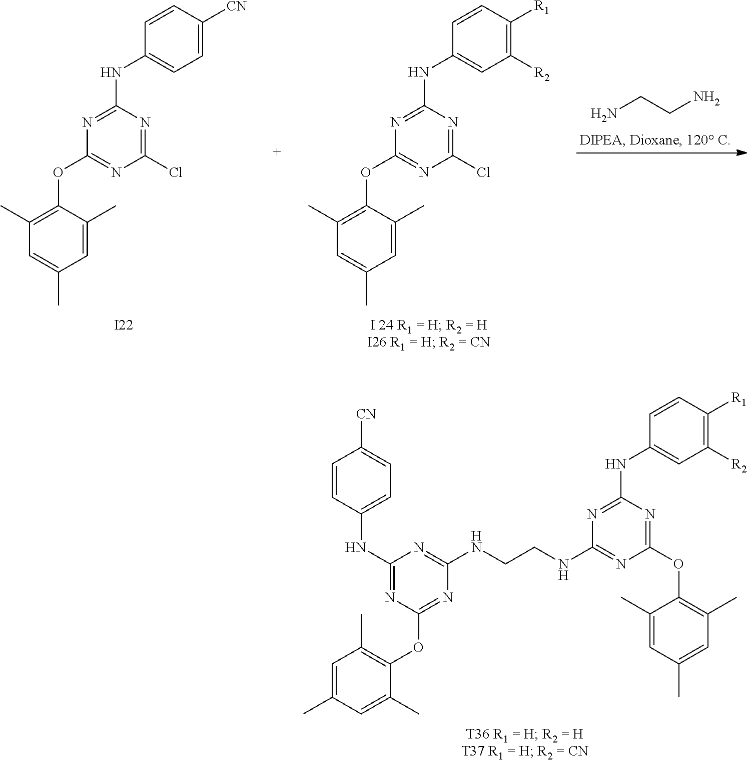

### 2,4-dichloro-6-(mesityloxy)-1,3,5-triazine (I21)

To a solution of 2,4,6-trichloro-1,3,5-triazine (2.77 g, 15 mmol) and 2,4,6-trimethylphenol (2.04 g, 15 mmol) and tetra-n-butyl ammonioum hydrogen sulfate (0.051 g, 0.15 mmol) in 50 mL of toluene at 0° C. was added slowly 5 mL of NaOH (0.6 g, 15 mmol) and allowed to stir at 0° C. for 2 h and then at RT for 36 h. EtOAc was added and washed with water, 10% HCl and brine. Removal of solvent afforded white solid (3.5 g, 82%)

¹H NMR (CDCl₃, 400 MHz) δ 6.97 (s, 2H), 2.31 (s, 3H), 2.10 (s, 6H); MS (ESI) m/z 285(M+H)+

### 4-((4-chloro-6-(mesityloxy)-1,3,5-triazin-2-yl)amino)benzonitrile (I22)

The above intermediate was prepared from I21 using the procedure similar to I4

Yield: 70%

¹H NMR (DMSO-d₆, 400 MHz) δ 11.19 (br s, 1H), 7.57 (br s, 4H), 6.95 (s, 2H), 2.30 (s, 3H), 2.06 (br s, 6H); MS (ESI) m/z 366(M+H)⁺; LC-MS (214 nm) tᵣ 19.7 min, 100%

### 4-((4-amino-6-(mesityloxy)-1,3,5-triazin-2-yl)amino)benzonitrile (I23)

The above intermediate was prepared from I22 using the procedure similar to I5.

Yield: 75%

¹H NMR (MeOD, 400 MHz) δ 7.74 (br s, 2H), 7.50 (s, 2H), 6.92 (s, 2H), 2.33 (br s, 3H), 2.13 (br s, 6H); MS (ESI) m/z 347(M+H)⁺; LC-MS (214 nm) tᵣ 18.0 min, 100%

### 4-chloro-6-(mesityloxy)-N-phenyl-1,3,5-triazin-2-amine (I24)

The above intermediate was prepared from I21 and aniline using the procedure similar to I4.

Yield: 71%

¹H NMR (MeOD, 400 MHz) δ 7.54 (br s, 2H), 7.27-6.91 (m, 5H), 2.30 (br s, 3H), 2.10 (br s, 6H); MS (ESI) m/z 341(M+H)⁺; LC-MS (214 nm) tᵣ 19.8 min, 100%.

### 6-(mesityloxy)-N₂-phenyl-1,3,5-triazine-2,4-diamine (I25)

The above intermediate was prepared from I24 using the procedure similar to I5.

Yield: 59%

¹H NMR (MeOD, 400 MHz) δ 7.47 (br s, 2H), 7.15 (br s, 2H), 6.90 (br s, 3H), 2.33 (br s, 3H), 2.09 (br s, 6H); MS (ESI) m/z 322(M+H)⁺; LC-MS (214 nm) tᵣ 18.1 min, 100%

### 3-((4-chloro-6-(mesityloxy)-1,3,5-triazin-2-yl)amino)benzonitrile (I26)

The above intermediate was prepared from I21 and 3-aminobenzonitrile using the procedure similar to I4.

Yield: 57%

¹H NMR (MeOD, 400 MHz) δ 7.72 (s, 1H), 7.44 (s, 1H), 7.31 (br s, 2H), 6.98 (s, 2H), 2.26 (br s, 3H), 2.1 (br s, 6H); MS (ESI) m/z 366 (M+H)⁺; LC-MS (214 nm) tᵣ 19.3 min, 93%

### 3-((4-amino-6-(mesityloxy)-1,3,5-triazin-2-yl)amino)benzonitrile (I27)

The above intermediate was prepared from I26 using the procedure similar to I5.

Yield: 55%

¹H NMR (MeOD, 400 MHz) δ 8.01 (br s, 1H), 7.6 (br s, 1H), 7.26 (br s, 2H), 6.91 (s, 2H), 2.23 (br s, 3H), 2.1 (br s, 6H); MS (ESI) m/z 347(M+H)⁺; LC-MS (214 nm) tᵣ 17.8 min, 100%

### 4,4′-((6,6′-(ethane-1,2-diylbis(azanediyl))bis(4-(mesityloxy)-1,3,5-triazine-6,2-diyl))bis(azanediyl))dibenzonitrile (T31)

The above compound was prepared from ethane-1,2-diamine and I22 using the procedure similar to T1

Yield: 52%

¹H NMR (MeOD, 400 MHz) δ 7.85 (br s, 4H), 7.56 (br s, 4H), 7.08 (br s, 4H), 3.74-3.81 (m, 4H), 2.47 (br s, 6H), 2.26 (br s, 12H); MS (ESI) m/z 720 (M+H)⁺; LC-MS (214 nm) tᵣ 22.4 min, 100%

### 4,4′-((6,6′-(propane-1,3-diylbis(azanediyl))bis(4-(mesityloxy)-1,3,5-triazine-6,2-diyl))bis(azanediyl))dibenzonitrile (T32)

The above compound was prepared from propane-1,3-diamine and I22 using the procedure similar to T1 Yield: 33%

¹H NMR (MeOD, 400 MHz) δ 7.7 (br s, 4H), 7.45 (br s, 4H), 6.88 (br s, 4H), 3.35-3.29 (m, 4H), 2.28 (br s, 6H), 2.00 (br s, 12H), 1.88 (br s, 2H); MS (ESI) m/z 734 (M+H)⁺; LC-MS (214 nm) tᵣ 23.1 min, 100%

### 4,4′-((6,6′-(pentane-1,5-diylbis(azanediyl))bis(4-(mesityloxy)-1,3,5-triazine-6,2-diyl))bis(azanediyl))dibenzonitrile (T33)

The above compound was prepared from pentane-1,5-diamine and I22 using the procedure similar to T1

Yield: 30%

¹H NMR (MeOD, 400 MHz) δ 7.73 (br s, 4H), 7.45 (br s, 4H), 6.88 (br s, 4H), 3.35-3.25 (m, 4H), 2.28 (br s, 6H), 2.06 (br s, 12H), 1.63 (br s, 6H); MS (ESI) m/z 762 (M+H)⁺; LC-MS (214 nm) tᵣ 23.1 min, 100%

### N₂,N₂-(ethane-1,2-diyl)bis(6-(mesityloxy)-N4-phenyl-1,3,5-triazine-2,4-diamine) (T34)

The above compound was prepared from ethane-1,2-diamine and I24 using the procedure similar to T1

Yield: 36%

¹H NMR (MeOD, 400 MHz) δ 7.46 (br s, 4H), 7.11 (br s, 4H), 6.86 (br s, 6H), 3.66-3.50 (m, 4H), 2.26 (br s, 6H), 2.06 (br s, 12H); MS (ESI) m/z 670 (M+H)⁺; LC-MS (214 nm) tᵣ 22.5 min, 100%

### 3,3′-((6,6′-(ethane-1,2-diylbis(azanediyl))bis(4-(mesityloxy)-1,3,5-triazine-6,2-diyl))bis(azanediyl))dibenzonitrile (T35)

The above compound was prepared from ethane-1,2-diamine and I26 using the procedure similar to T1

Yield: 39%

¹H NMR (MeOD, 400 MHz) δ 7.90 (br s, 2H), 7.55 (br s, 2H), 7.16 (br s, 4H), 6.83 (s, 4H), 3.69-3.54 (m, 4H), 2.24 (br s, 6H), 2.06 (br s, 12H); MS (ESI) m/z 720 (M+H)⁺; LC-MS (214 nm) tᵣ 22.2 min, 100%

### 3-((4-(mesityloxy)-6-((2-((4-(mesityloxy)-6-(phenylamino)-1,3,5-triazin-2-yl)amino)ethyl)amino)-1,3,5-triazin-2-yl)amino)benzonitrile (T36)

The above compound was prepared from intermediates I22, I24 and ethane-1,2-diamine using the procedure similar to T25

Yield: 25%

¹H NMR (MeOD, 400 MHz) δ 7.70 (br s, 2H), 7.39 (br s, 4H), 7.11 (br s, 2H), 6.88 (br s, 5H), 3.67-3.50 (m, 4H), 2.28 (br s, 6H), 2.07 (br s, 12H); MS (ESI) m/z 695 (M+H)⁺; LC-MS (214 nm) tᵣ 22.2 min, 100%

### 3-((4-((2-((4-((4-cyanophenyl)amino)-6-(mesityloxy)-1,3,5-triazin-2-yl)amino)ethyl)amino)-6-(mesityloxy)-1,3,5-triazin-2-yl)amino)benzonitrile (T37)

The above compound was prepared from intermediates I22, I26 and ethane-1,2-diamine using the procedure similar to T25

Yield: 35%

¹H NMR (MeOD, 400 MHz) δ 7.64 (br s, 4H), 7.36 (br s, 4H), 6.87 (br s, 4H), 3.68-3.60 (m, 4H), 2.27 (br s, 6H), 2.07 (br s, 12H); MS (ESI) m/z 720 (M+H)⁺; LC-MS (214 nm) tᵣ 22.0 min, 100%.

### Example 11

### Synthesis of intermediates I28-I36 and target compounds T38-T43

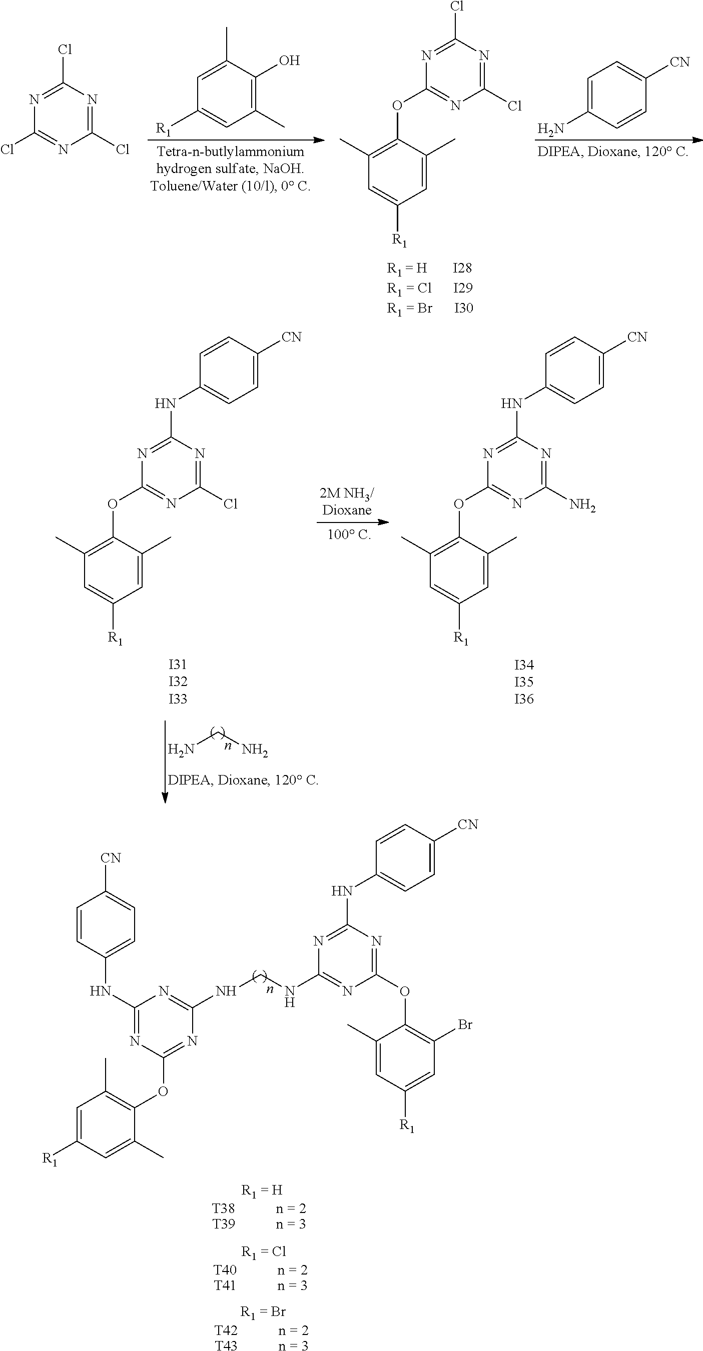

### 2,4-dichloro-6-(2,6-dimethylphenoxy)-1,3,5-triazine (I28)

The above intermediate was prepared from 2,4,6-trichloro-1,3,5-triazine and 2,6-dimethylphenol using the procedure similar to I21

Yield: 88%

### 2,4-dichloro-6-(4-chloro-2,6-dimethylphenoxy)-1,3,5-triazine (I29)

The above intermediate was prepared from 2,4,6-trichloro-1,3,5-triazine and 2,6-dimethyl-4-chlorophenol using the procedure similar to I21

Yield: 85%

¹H NMR (DMSO-d₆, 400 MHz) δ 7.25 (s, 2H), 2.12 (s, 6H)

### 2-(4-bromo-2,6-dimethylphenoxy)-4,6-dichloro-1,3,5-triazine (I30)

The above intermediate was prepared from 2,4,6-trichloro-1,3,5-triazine and 2,6-dibromo-4-bromophenol using the procedure similar to I21

Yield: 80%

¹H NMR (DMSO-d₆, 400 MHz) δ 7.36 (s, 2H), 2.11 (s, 6H)

### 4-((4-chloro-6-(2,6-dimethylphenoxy)-1,3,5-triazin-2-yl)amino)benzonitrile (I31)

The above intermediate was prepared from I28 using the procedure similar to I4.

Yield: 68%

¹H NMR (DMSO-d₆, 400 MHz) δ 8.0-7.2 (m, 7H), 2.10 (s, 6H); MS (ESI) m/z 353 (M+H)⁺; LC-MS (214 nm) tᵣ 18.7 min, 100%

### 4-((4-chloro-6-(4-chloro-2,6-dimethylphenoxy)-1,3,5-triazin-2-yl)amino)benzonitrile (I32)

The above intermediate was prepared from I29 using the procedure similar to I4.

Yield: 67%

¹H NMR (DMSO-d₆, 400 MHz) δ 7.8-7.2 (m, 6H), 2.05 (s, 6H); MS (ESI) m/z 387 (M+H)⁺; LC-MS (214 nm) tᵣ 19.6 min, 100%

### 4-((4-(4-bromo-2,6-dimethylphenoxy)-6-chloro-1,3,5-triazin-2-yl)amino)benzonitrile (I33)

The above intermediate was prepared from I30 using the procedure similar to I4.

Yield: 65%

¹H NMR (DMSO-d₆, 400 MHz) δ 8.0-7.45 (m, 6H), 2.10 (s, 6H); MS (ESI) m/z 432 (M+H)⁺; LC-MS (214 nm) tᵣ 19.8 min, 100%

### 4-((4-amino-6-(2,6-dimethylphenoxy)-1,3,5-triazin-2-yl)amino)benzonitrile (I34)

The above intermediate was prepared from I31 using the procedure similar to I5.

Yield: 62%

¹H NMR (DMSO-d₆, 400 MHz) δ 7.83 (br s, 2H), 7.60 (s, 2H), 7.33 (br s, 2H), 7.13-7.06 (m, 3H), 2.10 (s, 6H); MS (ESI) m/z 333 (M+H)⁺; LC-MS (214 nm) tᵣ 16.8 min, 100%

### 4-((4-amino-6-(4-chloro-2,6-dimethylphenoxy)-1,3,5-triazin-2-yl)amino)benzonitrile (I35)

The above intermediate was prepared from I32 using the procedure similar to I5.

Yield: 66%

¹H NMR (DMSO-d₆, 400 MHz) δ 7.84 (br s, 2H), 7.61 (s, 2H), 7.28 (br s, 2H), 7.18 (s, 2H), 2.03 (s, 6H); MS (ESI) m/z 368 (M+H)⁺; LC-MS (214 nm) tᵣ 18.0 min, 100%

### 4-((4-amino-6-(4-bromo-2,6-dimethylphenoxy)-1,3,5-triazin-2-yl)amino)benzonitrile (I36)

The above intermediate was prepared from I33 using the procedure similar to I5.

Yield: 63%

¹H NMR (DMSO-d₆, 400 MHz) δ 7.85 (br s, 2H), 7.61 (s, 2H), 7.37 (br s, 4H), 2.10 (s, 6H); MS (ESI) m/z 412 (M+H)⁺; LC-MS (214 nm) tᵣ 18.3 min, 100%

### 4,4′-((6,6′-(ethane-1,2-diylbis(azanediyl))bis(4-(2,6-dimethylphenoxy)-1,3,5-triazine-6,2-diyl))bis(azanediyl))dibenzonitrile (T38)

The above compound was prepared from ethane-1,2-diamine and I31 using the procedure similar to T1

Yield: 39%

¹H NMR (MeOD, 400 MHz) δ 7.64 (br s, 4H), 7.37 (br s, 4H), 7.14-7.0 (m, 6H), 3.47-3.3 (m, 4H), 2.16-2.03 (m, 12H); MS (ESI) m/z 692 (M+H)⁺; LC-MS (214 nm) tᵣ 20.8 min, 100

### 4,4′-((6,6′-(propane-1,3-diylbis(azanediyl))bis(4-(2,6-dimethylphenoxy)-1,3,5-triazine-6,2-diyl))bis(azanediyl))dibenzonitrile (T39)

The above compound was prepared from propane-1,3-diamine and I31 using the procedure similar to T1

Yield: 31%

¹H NMR (MeOD, 400 MHz) δ 7.67 (br s, 4H), 7.37 (br s, 4H), 7.14-7.05 (m, 6H), 3.54-3.35 (m, 4H), 2.12-2.04 (m, 12H), 1.77 (br s, 2H); MS (ESI) m/z 706 (M+H)⁺; LC-MS (214 nm) tᵣ 21.7 min, 100%

### 4,4′-((6,6′-(ethane-1,2-diylbis(azanediyl))bis(4-(4-chloro-2,6-dimethylphenoxy)-1,3,5-triazine-6,2-diyl))bis(azanediyl))dibenzonitrile (T40)

The above compound was prepared from ethane-1,2-diamine and I32 using the procedure similar to T1

Yield: 34%

¹H NMR (MeOD, 400 MHz) δ 7.69 (br s, 4H), 7.43 (br s, 4H), 7.15 (br s, 4H), 3.73-3.57 (m, 4H), 2.19-2.04 (m, 12H); MS (ESI) m/z 761 (M+H)⁺; LC-MS (214 nm) tᵣ 22.2 min, 100%

### 4,4′-((6,6′-(propane-1,3-diylbis(azanediyl))bis(4-(4-chloro-2,6-dimethylphenoxy)-1,3,5-triazine-6,2-diyl))bis(azanediyl))dibenzonitrile (T41)

The above compound was prepared from propane-1,3-diamine and I32 using the procedure similar to T1.

Yield: 31%

¹H NMR (MeOD, 400 MHz) δ 7.76-7.41 (m, 8H), 7.13-7.04 (m, 4H), 3.51-3.32 (m, 4H), 2.16-2.03 (m, 12H), 1.88 (br s, 2H); MS (ESI) m/z 775 (M+H)⁺; LC-MS (214 nm) tᵣ 22.9 min, 100%

### 4,4′-((6,6′-(ethane-1,2-diylbis(azanediyl))bis(4-(4-bromo-2,6-dimethylphenoxy)-1,3,5-triazine-6,2-diyl))bis(azanediyl))dibenzonitrile (T42)

The above compound was prepared from ethane-1,2-diamine and I33 using the procedure similar to T1

Yield: 47%

¹H NMR (MeOD, 400 MHz) δ 7.64 (br s, 4H), 7.44-7.37 (m, 4H), 7.25-7.15 (m, 4H), 3.69-3.53 (m, 4H), 2.15-2.04 (m, 12H); MS (ESI) m/z 850 (M+H)⁺; LC-MS (214 nm) tᵣ 22.5 min, 100%

### 4,4′-((6,6′-(propane-1,3-diylbis(azanediyl))bis(4-(4-bromo-2,6-dimethylphenoxy)-1,3,5-triazine-6,2-diyl))bis(azanediyl))dibenzonitrile (T43)

The above compound was prepared from propane-1,3-diamine and I33 using the procedure similar to T1.

Yield: 28%

¹H NMR (MeOD, 400 MHz) δ 7.69 (br s, 4H), 7.46 (br s, 4H), 7.24-7.2 (m, 4H), 3.52-3.35 (m, 4H), 2.3-2.06 (m, 12H), 1.87 (br s, 2H); MS (ESI) m/z 864 (M+H)⁺; LC-MS (214 nm) tᵣ 23.2 min, 100%

### Example 12

### Synthesis of Intermediates I37-I39 and Target Compounds T44 and T45

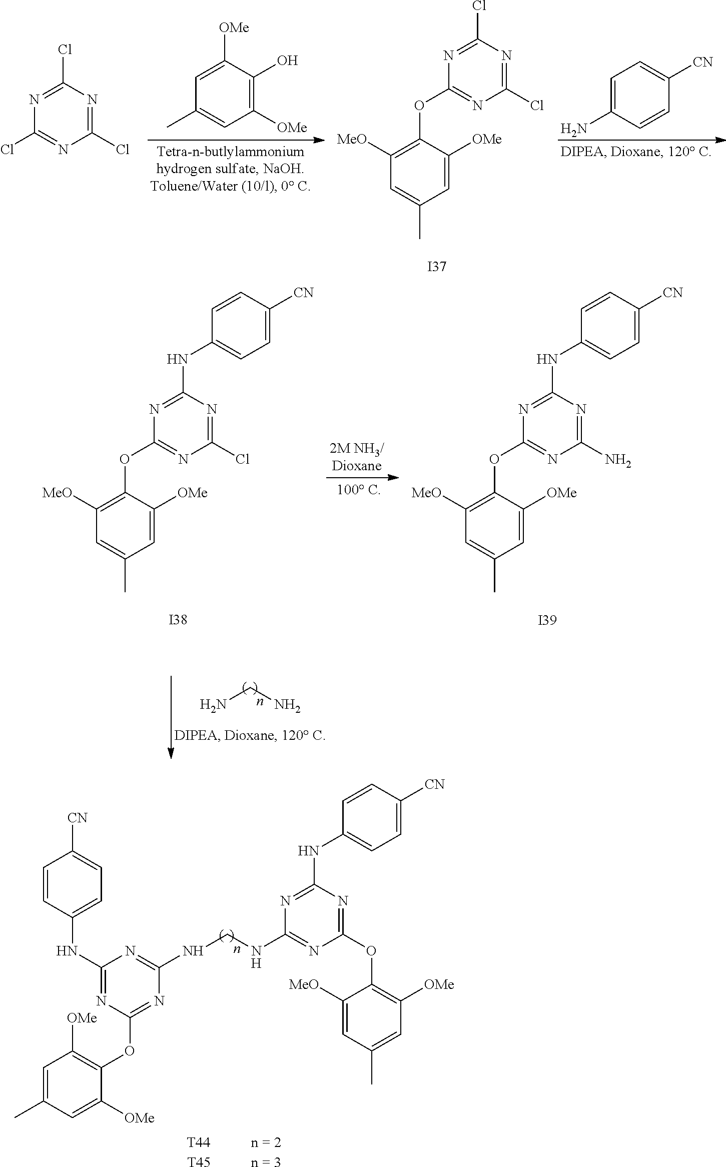

### 2,4-dichloro-6-(2,6-dimethoxy-4-methylphenoxy)-1,3,5-triazine (I37)

The above intermediate was prepared from 2,4,6-trichloro-1,3,5-triazine and 2,6-dimethoxy-4-methylphenol using the procedure similar to I21

Yield: 99%

¹H NMR (DMSO-d₆, 400 MHz) δ 6.55 (s, 2H), 3.79 (s, 6H), 2.3 (s, 3H)

### 4-((4-chloro-6-(2,6-dimethoxy-4-methylphenoxy)-1,3,5-triazin-2-yl)amino)benzonitrile (I38)

The above intermediate was prepared from I37 using the procedure similar to I4.

Yield: 45%

¹H NMR (DMSO-d₆, 400 MHz) δ 7.8 (br s, 4H), 6.71 (s, 2H), 3.72 (s, 6H), 2.41 (s, 3H); MS (ESI) m/z 399 (M+H)⁺; LC-MS (214 nm) tᵣ 18.0 min, 100%

### 4-((4-amino-6-(2,6-dimethoxy-4-methylphenoxy)-1,3,5-triazin-2-yl)amino)benzonitrile (I39)

The above intermediate was prepared from I38 using the procedure similar to I5.

Yield: 55%

¹H NMR (DMSO-d6, 400 MHz) δ 8.04 (s, 1H), 7.73 (br s, 2H), 7.65 (br s, 1H), 7.61 (br s, 2H), 7.26 (br s, 2H), 3.70 (s, 6H), 2.33 (s, 3H); MS (ESI) m/z 379 (M+H)⁺; LC-MS (214 nm) tᵣ 15.9 min, 100%

### 4,4′-((6,6′-(ethane-1,2-diylbis(azanediyl))bis(4-(2,6-dimethoxy-4-methylphenoxy)-1,3,5-triazine-6,2-diyl))bis(azanediyl))dibenzonitrile (T44)

The above compound was prepared from ethane-1,2-diamine and I38 using the procedure similar to T1

Yield: 41%

¹H NMR (DMSO-d6, 400 MHz) δ 7.85 (br s, 4H), 7.37 (br s, 4H), 6.57 (br s, 4H), 3.66 (br s, 12H), 3.52-3.48 (m, 4H), 2.38 (s, 6H); MS (ESI) m/z 784 (M+H)⁺; LC-MS (214 nm) tᵣ 19.5 min, 100%

### 4,4′-((6,6′-(propane-1,3-diylbis(azanediyl))bis(4-(2,6-dimethoxy-4-methylphenoxy)-1,3,5-triazine-6,2-diyl))bis(azanediyl))dibenzonitrile (T45)

The above compound was prepared from propane-1,3-diamine and I38 using the procedure similar to T1.

Yield: 22%

¹H NMR (MeOD, 400 MHz) δ 7.66 (br s, 4H), 7.43 (br s, 4H), 6.57 (br s, 4H), 3.72 (br s, 12H), 3.52-3.35 (m, 4H), 2.40 (br s, 6H), 1.86 (br s, 2H); MS (ESI) m/z 798 (M+H)⁺; LC-MS (214 nm) tᵣ 20.1 min, 100%

### Example 13

### Synthesis of Target Compound T46

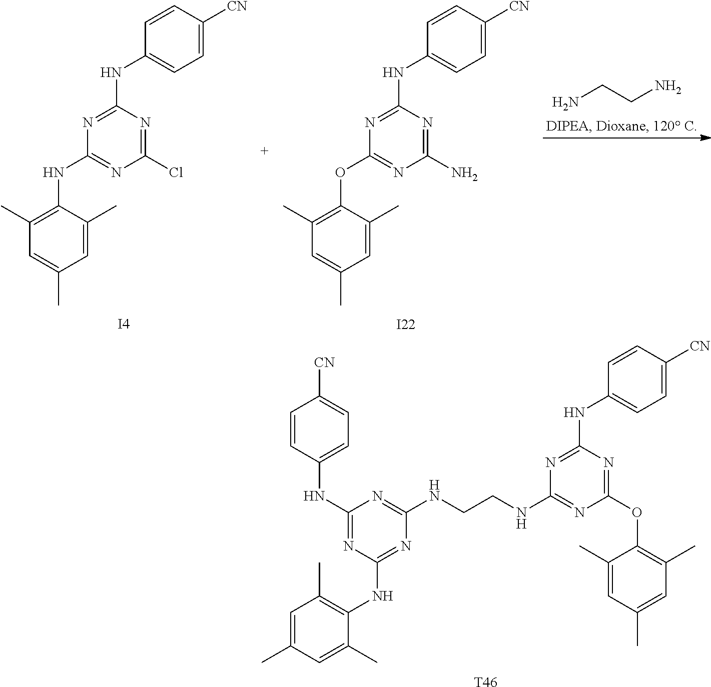

### 4-((4-((2-((4-((4-cyanophenyl)amino)-6-(mesitylamino)-1,3,5-triazin-2-yl)amino)ethyl)amino)-6-(mesityloxy)-1,3,5-triazin-2-yl)amino)benzonitrile (T46)

The above compound was prepared from intermediates I4, I22 and ethane-1,2-diamine using the procedure similar to T25

Yield: 51%

¹H NMR (MeOD, 400 MHz) δ 7.91 (br s, 1H), 7.59 (br s, 4H), 7.39 (br s, 3H), 6.90 (br s, 4H), 3.65 (br s, 4H), 2.3-2.0 (m, 18H); MS (ESI) m/z 719 (M+H)⁺; LC-MS (214 nm) tᵣ 21.6 min, 100%

### Assays

### Antiprotozoal Assays

The antiprotozoal assays were performed at the Laboratory of Microbiology, Parasitology and Hygiene (LMPH), Antwerp University, adopting the set of standard protocols as described by Cos et al. (10) and IC₅₀ values were determined from five 4-fold dilutions, starting from a maximum concentration of 64 μg/mL and data are reported as means±SD.

### Cytotoxicity Assay

Human lung fibroblast MRC-5SV2 cells were cultured in Earl's MEM, supplemented with 5% heat-inactivated FBS, 20 mM L-glutamine and 16.5 mM sodium bicarbonate. Assays were performed in 96-well microtiter plates, each well containing 1×104 cells. IC₅₀ values were determined from five 4-fold dilutions starting at 64 μg/mL. After incubation for 72 h in a humidified atmosphere (37° C., 5% CO₂) and addition of resazurin, the cell viability was assessed fluorimetrically (λₑₓ 550 nm, λₑₘ 590 nm). The results were expressed as % reduction in cell growth/viability compared to untreated control wells and IC₅₀ values were determined. Tamoxifen was included as reference drug.

In Vitro Anti-Trypanosoma brucei brucei Activity:

The suramin-sensitive strain Trypanosoma b. brucei Squib 427 was maintained in HMI-9-medium, supplemented with 10% heat-inactivated FBS. Assays were performed in 96-well microtiter plates, each well containing 10 μL of the extract dilution together with 190 μL of the parasite suspension (7×104 parasites/mL). After incubation in a humidified atmosphere (37° C., 5% CO₂) for 72 h, resazurin was added for another 24 h and parasite growth was assessed fluorimetrically (λₑₓ 550 nm, λₑₘ 590 nm). The results were expressed as % reduction in parasite growth/viability compared to control wells and IC₅₀ values were calculated from five 4-fold dilutions starting with 64 μg/mL. Suramin was included as reference drug.

In Vitro Anti-Trypanosoma brucei rhodesiense Activity

T. rhodesiense (strain STIB-900) was maintained in HMI-9-medium, supplemented with 10% heat-inactivated FBS. Assays were performed in 96-well microtiter plates, each well containing 10 μL of the extract dilution together with 190 μL of the parasite suspension (2×10⁴ parasites/mL). After incubation in a humidified atmosphere (37° C., 5% CO2) for 72 h, resazurin was added for another 6 h and parasite growth was assessed fluorimetrically (λₑₓ 550 nm, λₑₘ 590 nm). The results were expressed as % reduction in parasite growth/viability compared to control wells and IC₅₀ values were calculated from five 4-fold dilutions starting with 64 μg/mL. Suramin was included as reference drug.

In Vitro Anti-Trypanosoma cruzi Activity

The nifurtimox-sensitive Trypanosoma cruzi, Tulahuen CL2, β galactosidase strain was maintained in MRC-5SV2 cells in MEM medium, supplemented with 200 mM L-glutamine, 16.5 mM sodium bicarbonate and 5% heat-inactivated FBS. All cultures and assays were conducted under a humidified atmosphere (37° C., 5% CO₂). Assays were performed in 96-well microtiter plates, each well containing 10 μL of the watery extract dilutions together with 190 μL of MRC-5SV2 cell/parasite inoculum (2×104 cells/mL and 2×105 parasites/mL). After incubation for 168 h, parasite growth was compared to untreated-infected controls. Parasite burdens were assessed after adding the substrate: 50 μL/well of a stock solution containing 15.2 mg CPRG (chlorophenolred β-Dgalactopyranoside) and 250 μL Nonidet in 100 ml PBS. The change in color was measured spectrophotometrically at 540 nm after 4 h at 37° C. The results were expressed as reduction in parasite burdens compared to control wells and IC₅₀ values were calculated from five fourfold dilutions starting with 64 μg/mL. Benznidazole was included as reference drug.

In Vitro Anti-Leishmania infantum Activity

Leishmania infantum MHOM/MA (BE)/67 was maintained in the golden hamster and spleen amastigotes were collected for preparing infection inocula. Primary peritoneal mouse macrophages were used as host cells and collected 48 h after peritoneal stimulation with a 2% potato starch suspension. Assays were performed in 96-well microtiter plates, each well containing 10 μL of the extract dilutions together with 190 μL of macrophage/parasite inoculum (3×105 cells and 3×106 parasites/well in RPMI-1640+5% heat-inactivated FBS). After incubation for 120 h in a humidified atmosphere (37° C., 5% CO₂), total parasite burdens were microscopically assessed after Giemsa staining. The results were expressed as % reduction in parasite burden compared to untreated control wells and IC₅₀ values were calculated. The extracts were tested using five fourfold dilutions starting with 64 μg/mL. Miltefosine was included as reference drug.

In Vitro Anti-Plasmodium falciparum Activity

The chloroquine-sensitive GHANA (P.fal-GHA) strain of P. falciparum was maintained in RPMI-1640 supplemented with 0.37 mM hypoxanthine, 25 mM HEPES buffer, 25 mM sodium bicarbonate and 10% human 0+ serum together with 2-4% washed human 0+ erythrocytes (11). All cultures and assays were conducted under a humidified atmosphere (37° C., 4% CO₂, 3% O₂ and 93% N₂) with the assay being an adaptation of the procedure described by Desjardins et al. (12). Assays were performed in 96-well microtiter plates, each well containing 10 μL of the watery extract dilutions together with 190 μL of the malaria parasite inoculum (1% parasitemia, 2% hematocrit). After incubation for 72 h at 37° C., the plates were frozen and stored at −20° C. Upon thawing, 20 μL of each well was transferred into another plate together with 100 μL Malstat® reagent and 20 μL of a 1/1 mixture of PES (phenazine ethosulphate, 2 mg/mL) and NBT (Nitro Blue Tetrazolium Grade III, 0.1 mg/ml). The plates were kept in the dark for 2 h and change in color was measured spectrophotometrically at 655 nm. The results were expressed as % reduction in parasitemia compared to control wells. The extracts were tested using five 4-fold dilutions starting with 64 μg/mL. Chloroquine was included as reference drug.

### Antiviral Assay

### Cells

The JC53-BL cell line, also known as the TZM-bl cell line (NIH AIDS Research and Reference Reagent Program, Germantown, USA), was used for the evaluation of anti-HIV activity. TZM-bl cells were cultured in Dulbecco's Minimum Essential Medium (DMEM) (Lonza) containing 10% heat-inactivated FBS and 50 μg gentamycin/mL at 37° C. in a humidified 5% CO₂, 95% air environment. Twice a week the cells were treated with 0.25% trypsin—1 mM EDTA (Lonza) for 10 minutes. The resulting cell suspension was washed with an equivalent amount of TZM-bl medium and subsequently seeded in a T75 culture flask (Greiner Bio-One, Germany) at 10⁶ cells in 20 mL medium.

### TZM-bl Assay

The antiviral activity of the newly designed compounds was measured by pre-incubating ten thousand TZM-bl cells (at 10⁵ cells/mL in culture medium supplemented with 30 μg/mL DEAE dextran) in a 96-well plate for 30 minutes at 37° C., 5% CO₂ in the presence or absence of serial dilutions of the respective compound. Subsequently, 200 TCID₅₀ of HIV-1 BaL was added to each well and cultures were incubated for 48 hours before quantifying luciferase activity, using a TriStar LB941 luminometer (Berthold Technologies GmbH & Co. KG, Bad Wildbad, Germany). Each condition was evaluated in triplicate wells and in at least two independent experiments. The antiviral activity of the compound was expressed as the percentage of viral inhibition compared to the untreated controls and subsequently plotted against the compound concentration. Non-linear regression analysis was used to calculate the 50% effective concentration (EC₅₀) based on at least two independent measurements and using GraphPad Prism version 5.03 for Windows (GraphPad Software, San Diego, Calif., USA).

### WST-1 Cytotoxicity Assay

The Water Soluble Tetrazolium-1 (WST-1) Cell Proliferation Assay is a colorimetric assay for the measurement of cell proliferation and viability. The assay is based on the cleavage of the tetrazolium salt WST-1 (4-[3-(4-iodophenyl)-2-(4-nitrophenyl)-2H-5-tetrazolio]-1,3-benzene disulfonate) to a formazan dye by a complex cellular mechanism. This bioreduction is largely dependent on the glycolytic production of NAD(P)H in viable cells. Therefore, the amount of formazan dye formed correlates directly to the number of viable cells in the culture, and can be quantified by measuring the absorbance at 450 nm in a multiwell plate reader. The greater the number of viable cells, the greater the amount of formazan dye produced following the addition of WST-1. Cytotoxicity of each compound was evaluated using this WST-1 viability assay, according to the manufacturer's instructions (Roche, Vilvoorde, Belgium).

Briefly, ten thousand TZM-bl cells were seeded in a 96-well plate and cultured for 2 days in the presence of a serial dilution of compound. After this 48 h exposure, Cell Proliferation Reagent, WST-1, was added and absorbance at 450 nm was quantified after 90 min using a microplate reader (BioRad, Tokio, Japan). Each compound was tested in three replicate wells and in at least two independent experiments. The percentage cell viability, compared to untreated controls, was plotted against the compound concentration and non-linear regression analysis was performed using GraphPad Prism version 5.02 for Windows (GraphPad Software, San Diego, Calif., USA) to calculate the 50% cytotoxic concentration (CC₅₀).

### Antiprotozoal Activity

The activity of intermediates (monomers) and target compounds (dimers) toward T. b. brucei, T. b. rhodesiense, T. cruzi, L. infantium, P. falciparum and their cytotoxicity on a human cell line (MRC5) is presented in Tables 1 and 2. Most of the target compounds (dimers, Table 2) possess high nanomolar activity and the best compound obtained in this series is T35 (IC₅₀=0.001 μM for T. brucei and 0.01 μM for T. rhodesiense). In addition no cytotoxicity (IC₅₀=>64 μM) is observed for all the target compounds.

TABLE 1
Antiprotozoal activity (IC₅₀ in μM) of intermediates (monomers)

| Cpd | T. bruc. | T. rhod. | T. cruz. | L. inf. | Pf-K1 | MRC-5 |
|---|---|---|---|---|---|---|
| I2 | 2.33 | 0.58 | >64 | >64 | >64 | >64 |
| I5 | 4.84 | 2.49 | 11.51 | 38.16 | 20.71 | 20.51 |
| I8 | 0.12 | 0.08 | 6.65 | >64.00 | 17.69 | 16.61 |
| I11 | 4.47 | 30.82 | >64 | >64 | >64 | >64 |
| I14 | 16.28 | 8.68 | 8.66 | 41.00 | 6.62 | >64 |
| I17 | 7.01 | 7.5 | 4.87 | 9.83 | 25.8 | 7.51 |
| I20 | 4.47 | 1.45 | 7.45 | 10.08 | 9.79 | 23.57 |
| I23 | 1.17 | 0.59 | 7.37 | >64 | >64 | 7.48 |
| I25 | 0.015 | 0.24 | >64 | 16.7 | 31.27 | >64 |
| I27 | 0.21 | 0.84 | 4.51 | 18.16 | 6.27 | 25.92 |
| I34 | 1.13 | 1.15 | 8.69 | 10.08 | >64 | >64 |
| I35 | 0.52 | 0.35 | 6.80 | 22.18 | 20.54 | 7.63 |
| I36 | 0.78 | 0.46 | 7.15 | 26.16 | 16.96 | 5.25 |
| I39 | 0.79 | 0.71 | 14.98 | 23.10 | 16.61 | 34.05 |

TABLE 2
Antiprotozoal activity (IC₅₀ in μM) of target compounds (dimers) T1-T46

| Cpd | T. bruc. | T. rhod. | T. cruz. | L. inf. | Pf-K1 | MRC-5 |
|---|---|---|---|---|---|---|
| T1 | 33.19 | 0.21 | >64 | 27.27 | 0.55 | >64 |
| T2 | 31.51 | 0.65 | >64 | >64 | 32.90 | >64 |
| T3 | >64 | 39.01 | >64 | >64 | 18.66 | >64 |
| T4 | >64 | 41.90 | >64 | 27.27 | >64 | >64 |
| T5 | 0.07 | 0.05 | >64 | >64 | 0.27 | >64 |
| T6 | 0.08 | 0.16 | >64 | 20.32 | 1.26 | >64 |
| T7 | 1.21 | 0.25 | >64 | 29.98 | 1.11 | >64 |
| T8 | 13.52 | 0.18 | >64 | >64 | 0.97 | >64 |
| T9 | 20.48 | 0.46 | >64 | >64 | 2.59 | >64 |
| T10 | 32.46 | 6.86 | >64 | >64 | 10.06 | >64 |
| T11 | 0.29 | 0.06 | >64 | >64 | 27.21 | >64 |
| T12 | 0.53 | 0.02 | >64 | >64 | >64 | >64 |
| T13 | 0.03 | 0.007 | >64 | 20.29 | 3.34 | >64 |
| T14 | 1.16 | 0.3 | >64 | 37.3 | 3.37 | >64 |
| T15 | 2.14 | 1.42 | >64 | 22.26 | 2.4 | >64 |
| T16 | 0.016 | 0.23 | >64 | 8 | 0.54 | >64 |
| T17 | 0.14 | 0.21 | 8.82 | 27.89 | 2.47 | >64 |
| T18 | 0.51 | 0.42 | 49.05 | >64 | >64 | >64 |
| T19 | 0.04 | 0.25 | 2.47 | 0.09 | 0.22 | >64 |
| T20 | 0.04 | 0.08 | 5.41 | 3 | 0.29 | >64 |
| T21 | 0.3 | 0.47 | >64 | 13.04 | 0.15 | >64 |
| T22 | 0.13 | 0.35 | >64 | 25.34 | 0.36 | >64 |
| T23 | 0.13 | 0.56 | >64 | 9.04 | 0.59 | >64 |
| T24 | 30.22 | 8.29 | >64 | 26.16 | 12.23 | >64 |
| T25 | 0.29 | 0.04 | 0.59 | >64 | 0.36 | >64 |
| T26 | 6.03 | 0.4 | >64 | >64 | >64 | >64 |
| T27 | 0.04 | 0.03 | >64 | >64 | 0.72 | >64 |
| T28 | 0.13 | 0.10 | >64 | >64 | 2.85 | >64 |
| T29 | 0.13 | 0.04 | >64 | 25.4 | 2.31 | >64 |
| T30 | 0.03 | 0.03 | >64 | >64 | 3.05 | >64 |
| T31 | 0.015 | 0.01 | >64 | 32.00 | 2.77 | >64 |
| T32 | 0.01 | 0.01 | >64 | >64 | 1.01 | >64 |
| T33 | 0.01 | 0.015 | >64 | 25.66 | 4.32 | >64 |
| T34 | 0.05 | 0.16 | 23.54 | 36.16 | 0.28 | >64 |
| T35 | 0.001 | 0.01 | 17.08 | 12 | 0.31 | >64 |
| T36 | 0.01 | 0.05 | >64 | 13.04 | 1.39 | >64 |
| T37 | 0.06 | 0.02 | >64 | 9.04 | 0.37 | >64 |
| T38 | 0.03 | 0.01 | 6.89 | 4.42 | 1.15 | >64 |
| T39 | 0.08 | 0.02 | 1.73 | 2.41 | 0.17 | >64 |
| T40 | 0.02 | 0.01 | 4.68 | 5.26 | 1.34 | >64 |
| T41 | 0.13 | 0.08 | 1.77 | 1.88 | 0.99 | >64 |
| T42 | 0.01 | 0.02 | >64 | 10.08 | 5.02 | >64 |
| T43 | 0.02 | 0.02 | 2.5 | 1.97 | 0.41 | >64 |
| T44 | 0.13 | 0.08 | >64 | 20.32 | 0.61 | >64 |
| T45 | 0.13 | 0.09 | >64 | >64 | 0.11 | >64 |
| T46 | 0.035 | 0.02 | 8.32 | 19.31 | 0.86 | >64 |

The comparison of the antiprotozoal activity of dimers vs monomers is shown in Tables 3-16. As a representative example the comparison of activity and cytotoxicity for dimers and their corresponding monomer in Table 3 is discussed below.

TABLE 3
Comparison of antiprotozoal activity (IC₅₀ in μM) of dimers T5, T6, T11 and T12 and
their corresponding monomer I5

| Cpd | Linker (L) | T. bruc. | T. rhod. | T. cruz. | L. inf | Pf-K1 | MRC-5 |
|---|---|---|---|---|---|---|---|
| I5 | — | 4.84 | 2.49 | 11.51 | 38.16 | 20.71 | 20.51 |
| T5 | NH(CH₂)₂NH | 0.07 | 0.05 | >64 | >64 | 0.27 | >64 |
| T6 | NH(CH₂)₃NH | 0.08 | 0.16 | >64 | 20.32 | 1.26 | >64 |
| T11 | 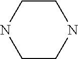 | 0.29 | 0.06 | >64 | >64 | 27.21 | >64 |
| T12 | 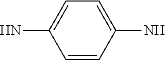 | 0.53 | 0.02 | >64 | >64 | >64 | >64 |

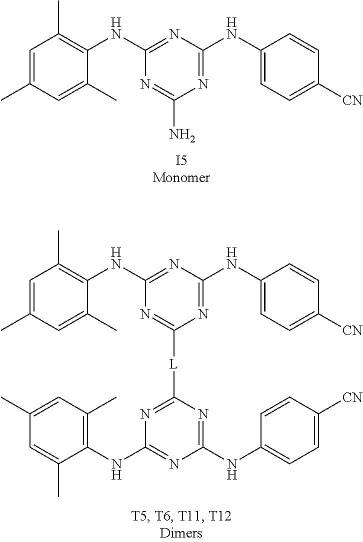

Monomer I5 is moderately active against T. b. brucei (IC₅₀=4.84 μM) and T. b. rhodesiense (IC₅₀=2.49 μM) and cytotoxic at 20.5 μM. Dimers T5, T6, T11 and T12 (obtained from monomer I5 with different organic linkers) show increase in trypanosomal activity and decrease in cytotoxicity in comparison with monomer I5.

Dimers T5, T6, T11 and T12 demonstrate 10-70 fold increase in T. brucei activity and 15-125 fold increase in T. rhodesiense activity and no cytotoxicity in comparison with monomer I5.

TABLE 4
Comparison of antiprotozoal activity (IC₅₀ in μM) of dimer T13 and its
corresponding monomer I8

| Cpd | Linker | T. bruc. | T. rhod. | T. cruz. | L. inf. | Pf-K1 | MRC-5 |
|---|---|---|---|---|---|---|---|
| I8 | — | 0.12 | 0.08 | 6.65 | >64.00 | 17.69 | 16.61 |
| T13 | NH(CH₂)₃NH | 0.03 | 0.007 | >64 | 20.29 | 3.34 | >64 |

TABLE 5
Comparison of antiprotozoal activity (IC₅₀ in μM) of dimer T16-T18 and its
corresponding monomer I11

| Cpd | Linker | T. bruc. | T. rhod. | T. cruz. | L. inf. | Pf-K1 | MRC-5 |
|---|---|---|---|---|---|---|---|
| I11 | — | 4.47 | 30.82 | >64 | >64 | >64 | >64 |
| T16 | NH(CH₂)₂NH | 0.016 | 0.23 | >64 | 8 | 0.54 | >64 |
| T17 | NH(CH₂)₃NH | 0.14 | 0.21 | 8.82 | 27.89 | 2.47 | >64 |
| T18 | NH(CH₂)₅NH | 0.51 | 0.42 | 49.05 | >64 | >64 | >64 |

TABLE 6
Comparison of antiprotozoal activity (IC₅₀ in μM) of dimers T19-T21 and their
corresponding monomer I14

| Cpd | Linker | T. bruc. | T. rhod. | T. cruz. | L. inf. | Pf-K1 | MRC-5 |
|---|---|---|---|---|---|---|---|
| I14 | — | 16.28 | 8.68 | 8.66 | 41.00 | 6.62 | >64 |
| T19 | NH(CH₂)₂NH | 0.04 | 0.25 | 2.47 | 0.09 | 0.22 | >64 |
| T20 | NH(CH₂)₃NH | 0.04 | 0.08 | 5.41 | 3 | 0.29 | >64 |
| T21 | NH(CH₂)₅NH | 0.3 | 0.47 | >64 | 13.04 | 0.15 | >64 |

TABLE 7
Comparison of antiprotozoal activity (IC₅₀ in μM) of dimers T22 and T23 and their
corresponding monomer I17

| Cpd | Linker | T. bruc. | T. rhod. | T. cruz. | L. inf. | Pf-K1 | MRC-5 |
|---|---|---|---|---|---|---|---|
| I17 | — | 7.01 | 7.5 | 4.87 | 9.83 | 25.8 | 7.51 |
| T22 | NH(CH₂)₂NH | 0.13 | 0.35 | >64 | 25.34 | 0.36 | >64 |
| T23 | NH(CH₂)₃NH | 0.13 | 0.56 | >64 | 9.04 | 0.59 | >64 |

TABLE 8
Comparison of antiprotozoal activity (IC₅₀ in μM) of dimer T25 and its
corresponding monomers I5 and I8

| Cpd | Linker | T. bruc. | T. rhod. | T. cruz. | L. inf. | Pf-K1 | MRC-5 |
|---|---|---|---|---|---|---|---|
| I5 | — | 4.84 | 2.49 | 11.51 | 38.16 | 20.71 | 20.51 |
| I8 | — | 0.12 | 0.08 | 6.65 | >64 | 17.69 | 16.61 |
| T25 | NH(CH₂)₂NH | 0.29 | 0.04 | 0.59 | >64 | 0.36 | >64 |

TABLE 9
Comparison of antiprotozoal activity (IC₅₀ in μM) of dimers T27 and T28 and their
corresponding monomers I2 and I5

| Cpd | Linker | T. bruc. | T. rhod. | T. cruz. | L. inf. | Pf-K1 | MRC-5 |
|---|---|---|---|---|---|---|---|
| I2 | — | 2.33 | 0.58 | >64 | >64 | >64 | >64 |
| I5 | — | 4.84 | 2.49 | 11.51 | 38.16 | 20.71 | 20.51 |
| T27 | NH(CH₂)₂NH | 0.04 | 0.03 | >64 | >64 | 0.72 | >64 |
| T28 | NH(CH₂)₃NH | 0.13 | 0.10 | >64 | >64 | 2.85 | >64 |

TABLE 10
Comparison of antiprotozoal activity (IC₅₀ in μM) of dimers T29 and T30 and their
corresponding monomer I20

| Cpd | Linker | T. bruc. | T. rhod. | T. cruz. | L. inf. | Pf-K1 | MRC-5 |
|---|---|---|---|---|---|---|---|
| I20 | — | 4.47 | 1.45 | 7.45 | 10.08 | 9.79 | 23.57 |
| T29 | NH(CH₂)₂NH | 0.13 | 0.04 | >64 | 25.4 | 2.31 | >64 |
| T30 | NH(CH₂)₃NH | 0.03 | 0.03 | >64 | >64 | 3.05 | >64 |

TABLE 11
Comparison of antiprotozoal activity (IC₅₀ in μM) of dimers T31-T33 and their
corresponding monomer I23

| Cpd | Linker | T. bruc. | T. rhod. | T. cruz. | L. inf. | Pf-K1 | MRC-5 |
|---|---|---|---|---|---|---|---|
| I23 | — | 1.17 | 0.59 | 7.37 | >64 | >64 | 7.48 |
| T31 | NH(CH₂)₂NH | 0.015 | 0.01 | >64 | 32 | 2.77 | >64 |
| T32 | NH(CH₂)₃NH | 0.01 | 0.01 | >64 | >64 | 1.01 | >64 |
| T33 | NH(CH₂)₅NH | 0.01 | 0.015 | >64 | 25.66 | 4.32 | >64 |

TABLE 12
Comparison of antiprotozoal activity (IC₅₀ in μM) of dimers T34 and T35 and their
corresponding monomers I25 and I27

| Cpd | Linker | T. bruc. | T. rhod. | T. cruz. | L. inf. | Pf-K1 | MRC-5 |
|---|---|---|---|---|---|---|---|
| I25 | — | 0.015 | 0.24 | >64 | 16.7 | 31.27 | >64 |
| I27 | — | 0.21 | 0.84 | 4.51 | 18.16 | 6.27 | 25.92 |
| T34 | NH(CH₂)₂NH | 0.05 | 0.16 | 23.54 | 36.16 | 0.28 | >64 |
| T35 | NH(CH₂)₂NH | 0.001 | 0.01 | 17.08 | 12 | 0.31 | >64 |

TABLE 13
Comparison of antiprotozoal activity (IC₅₀ in μM) of dimers T36 and T37 and their
corresponding monomers I23, I25 and I27

| Cpd | Linker | T. bruc. | T. rhod. | T. cruz. | L. inf. | Pf-K1 | MRC-5 |
|---|---|---|---|---|---|---|---|
| I23 | — | 1.17 | 0.59 | 7.37 | >64 | >64 | 7.48 |
| I25 | — | 0.015 | 0.24 | >64 | 16.7 | 31.27 | >64 |
| I27 | — | 0.21 | 0.84 | 4.51 | 18.16 | 6.27 | 25.92 |
| T36 | NH(CH₂)₂NH | 0.01 | 0.05 | >64 | 13.04 | 1.39 | >64 |
| T37 | NH(CH₂)₂NH | 0.06 | 0.02 | >64 | 9.04 | 0.37 | >64 |

TABLE 14
Comparison of antiprotozoal activity (IC₅₀ in μM) of dimers T38-T43 and their
corresponding monomers I34-I36

| Cpd | Linker | T. bruc. | T. rhod. | T. cruz. | L. inf. | Pf-K1 | MRC-5 |
|---|---|---|---|---|---|---|---|
| I34 | — | 1.13 | 1.15 | 8.69 | 10.08 | >64 | >64 |
| I35 | — | 0.52 | 0.35 | 6.80 | 22.18 | 20.54 | 7.63 |
| I36 | — | 0.78 | 0.46 | 7.15 | 26.16 | 16.96 | 5.25 |
| T38 | NH(CH₂)₂NH | 0.03 | 0.01 | 6.89 | 4.42 | 1.15 | >64 |
| T39 | NH(CH₂)₃NH | 0.08 | 0.02 | 1.73 | 2.41 | 0.17 | >64 |
| T40 | NH(CH₂)₂NH | 0.02 | 0.01 | 4.68 | 5.26 | 1.34 | >64 |
| T41 | NH(CH₂)₃NH | 0.13 | 0.08 | 1.77 | 1.88 | 0.99 | >64 |
| T42 | NH(CH₂)₂NH | 0.01 | 0.02 | >64 | 10.08 | 5.02 | >64 |
| T43 | NH(CH₂)₃NH | 0.02 | 0.02 | 2.5 | 1.97 | 0.41 | >64 |

TABLE 15
Comparison of antiprotozoal activity (IC₅₀ in μM) of dimers T44 and T45 and their
corresponding monomer I39

| Cpd | Linker | T. bruc. | T. rhod. | T. cruz. | L. inf. | Pf-K1 | MRC-5 |
|---|---|---|---|---|---|---|---|
| I39 | — | 0.79 | 0.28 | 14.98 | 23.10 | 16.61 | 34.05 |
| T44 | NH(CH₂)₂NH | 0.13 | 0.08 | >64 | 20.32 | 0.61 | >64 |
| T45 | NH(CH₂)₃NH | 0.13 | 0.09 | >64 | >64 | 0.11 | >64 |

TABLE 16
Comparison of antiprotozoal activity (IC₅₀ in μM) of dimer T46 and their
corresponding monomers I5 and I23

| Cpd | Linker | T. bruc. | T. rhod. | T. cruz. | L. inf. | Pf-K1 | MRC-5 |
|---|---|---|---|---|---|---|---|
| I5 | — | 4.84 | 2.49 | 11.51 | 38.16 | 20.71 | 20.51 |
| I23 | — | 1.17 | 0.59 | 7.37 | >64 | >64 | 7.48 |
| T46 | NH(CH₂)₂NH | 0.035 | 0.02 | 8.32 | 19.31 | 0.86 | >64 |

The anti-malarial activity of selected target compounds is presented in Table 17. Several compounds exhibit sub-micromolar activity and the best compound obtained is T45 (IC₅₀=0.11 μM).

TABLE 17
Selected anti-malarialactivity (IC₅₀ in μM) of dimers

| Cpd | Pf-K1 | MRC-5 |
|---|---|---|
| T1 | 0.55 | >64 |
| T5 | 0.27 | >64 |
| T8 | 0.97 | >64 |
| T16 | 0.54 | >64 |
| T19 | 0.22 | >64 |
| T20 | 0.29 | >64 |
| T21 | 0.15 | >64 |
| T22 | 0.36 | >64 |
| T23 | 0.59 | >64 |
| T25 | 0.36 | >64 |
| T27 | 0.72 | >64 |
| T34 | 0.28 | >64 |
| T35 | 0.31 | >64 |
| T37 | 0.37 | >64 |
| T39 | 0.17 | >64 |
| T43 | 0.41 | >64 |
| T44 | 0.61 | >64 |
| T45 | 0.11 | >64 |
| T46 | 0.86 | >64 |

### Anti-HIV-1 Activity

The target compounds are evaluated for their anti-HIV-1 activity and cytotoxicity in TZM-bl cells. The results, expressed as EC₅₀ (50% effective concentration), CC₅₀ (50% cytotoxic concentration) and SI (selectivity index given by the CC₅₀/EC₅₀ ratio) values are summarizedin Table 18. Several compounds exhibit sub-micromolar activity and the best compounds obtained in this series are T5, T23 and T24 (EC₅₀=<0.15 μm) and no cytotoxicity is observed for most of the target compounds (EC₅₀=>100 μm).

TABLE 18
Anti-HIV-1 activity of dimers T1-T46

| Cpd | EC₅₀ μm | CC₅₀ μm | SI |
|---|---|---|---|
| T1 | >10 | nd | — |
| T2 | >10 | nd | — |
| T3 | >10 | nd | — |
| T4 | >10 | nd | — |
| T5 | 0.151 | >100 | >664 |
| T6 | 0.203 | >100 | >493 |
| T7 | 1.487 | >100 | >67 |
| T8 | 0.710 | >100 | >141 |
| T9 | 0.872 | >100 | >115 |
| T10 | 0.268 | >100 | >374 |
| T11 | 6.074 | >100 | >16 |
| T12 | 0.167 | >100 | >599 |
| T13 | 0.592 | >100 | >168 |
| T14 | 0.460 | >100 | >217 |
| T15 | 1.628 | >100 | >61 |
| T16 | >10 | nd | — |
| T17 | 0.249 | >100 | >402 |
| T18 | 0.488 | >100 | >205 |
| T19 | 1.438 | >100 | >70 |
| T20 | 0.566 | >100 | >176 |
| T21 | 0.700 | >100 | >143 |
| T22 | 0.199 | >100 | >501 |
| T23 | 0.136 | >100 | >736 |
| T24 | 0.143 | >100 | >697 |
| T25 | 2.712 | >100 | >37 |
| T26 | 3.191 | >100 | >31 |
| T27 | 7.978 | >100 | >12 |
| T28 | 0.562 | >100 | >177 |
| T29 | 0.573 | >100 | >174 |
| T30 | 0.738 | >100 | >135 |
| T31 | 1.277 | >100 | >78 |
| T32 | 0.816 | >100 | >123 |
| T33 | 0.967 | >100 | >103 |
| T34 | >10 | nd | --- |
| T35 | >10 | nd | --- |
| T36 | 0.376 | >100 | >266 |
| T37 | 0.723 | >100 | >138 |
| T38 | 4.233 | 49.514 | 12 |
| T39 | 5.153 | 48.624 | 9 |
| T40 | 0.919 | >100 | >108 |
| T41 | 0.655 | 30.099 | 46 |
| T42 | 1.127 | >100 | >88 |
| T43 | 0.423 | 21.281 | 50 |
| T44 | 0.755 | >100 | >132 |
| T45 | >10 | nd | — |
| T46 | 3.633 | >100 | >28 |

### REFERENCES

1. Coinfection with HIV and tropical infectious diseases. I. Protozoal pathogens. Karp, C. L.; Auwaerter, P. G. Clin. Infect. Dis. 2007, 45, 1208-1213.
2. HIV-1/parasite co-infection and the emergence of new parasite strains. Lloyd-Smith, J. O.; Poss, M.; Grenfell, B. T. Parasitology 2008, 135, 795-806.
3. Manifestations of Chagas disease (American trypanosomiasis) in patients with HIV/AIDS. Sartori, A. M.; Ibrahim, K. Y.; Nunes Westphalen, E. V.; Braz, L. M.; Oliveira, O. C., Jr.; Gakiya, E.; Lopes, M. H.; Shikanai-Yasuda, M. A. Ann. Trop. Med. Parasitol. 2007, 101, 31-50.
4. Evolution of anti-HIV drug candidates. Part 2: Diaryltriazine (DATA) analogues. Ludovici, D. W.; Kavash, R. W.; Kukla, M. J.; Ho, C. Y.; Ye, H.; De Corte, B. L.; Andries, K.; de Bethune, M. P.; Azijn, H.; Pauwels, R.; Moereels, H. E.; Heeres, J.; Koymans, L. M.; de Jonge, M. R.; Van Aken, K. J.; Daeyaert, F. F.; Lewi, P. J.; Das, K.; Arnold, E.; Janssen, P. A. Bioorg. Med. Chem. Lett. 2001, 11, 2229-34.
5. Trisubstituted 1,3,5-triazine derivatives for treatment of HIV infections. Daeyaert, F. F. D.; De Corte, B.; De Jonge, M. R.; Heeres, J.; Ho, C. Y.; Janssen, P. A. J.; Kavash, R. W.; Koymans, L. M. H.; Kukla, M. J.; Ludovici, D. W. WO 99/50256.
6. Trisubstituted 1,3,5-triazine derivatives for treatment of HIV infections. Kukla, M. J.; Ludovici, D. W.; Kavash, R. W.; Heeres, J.; Janssen, P. A. J. EP 0 945 447 A1.
7. HIV replication inhibitors. Kukla, M. J.; Ludovici, D. W.; Kavash, R. W.; De Corte, B. L. D.; Heeres, J.; Janssen, P. A. J.; Koymans, L. M. H.; De Jonge, M. R.; Van Aken, K. J. A.; Krief, A.; Leenders, R. G. G. WO 2001/85700.
8. HIV replication inhibiting pyrimidines and triazines. Guillemont, J. E. G.; Pasquier, E. T, J.; Heeres, J.; Hertogs, K.; Bettens, E.; Lewi, P. J.; De Jonge, M. R. Koymans, L. M. H.; Daeyaert, F. F. D.; Vinkers, H. M. WO 2004/074262.
9. Microbicidal pyrimidine or triazine for preventing sexual HIV transmission. Van Roey, J. M.; De Bethune, M. T. M. M. G.; Stoffels, P. WO 2003/094920 and US 2011/0165093.
10. Anti-infective potential of natural products: how to develop a stronger in vitro ‘proof-of-concept’. Cos P, Vlietinck A J, Berghe D V, Maes L. J. Ethnopharmacol. 2006, 106, 290-302.
11. Continuous culture of Plasmodium falciparum: its impact on malaria research Trager W, Jensen J B. Int. J. Parasitol. 1997, 27, 989-1006.
12. Quantitative assessment of antimalarial activity in vitro by a semiautomated microdilution technique. Desjardins R E, Canfield C J, Haynes J D, Chulay J D. Antimicrob. Agents Chemother. 1979, 167, 10-8.

1.-15. (canceled)

16. A compound of formula I or a stereoisomer, tautomer, racemic, metabolite, pro- or predrug, salt, hydrate, or solvate thereof, 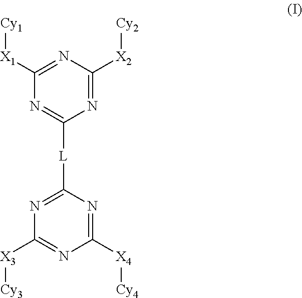
Wherein
X₁, X₂, X₃, and X₄ are independently selected from the group consisting of —NRₐ—, —O—, —S—, and —CHR_{b}—;
Rₐ and R_{b} are each independently selected from H and alkyl;
Cy₁, Cy₂, Cy₃ and Cy₄ are each phenyl; wherein said phenyl is optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group consisting of haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula 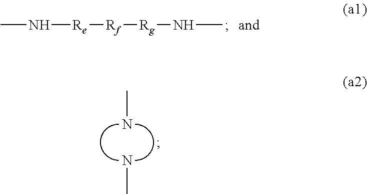
wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group consisting of aryl, heteroaryl, cycoalkyl, and heterocyclyl;
wherein —Rₑ—R_{f}—R_{g}— is not a direct bond; and wherein at least one of Cy₁, Cy₂, Cy₃, or Cy₄, is substituted with two or more substituents,
provided that said compound is not
1,2-Ethanediamine,N,N′-bis[4,6-bis(2,6-dimethylphenoxy)-1,3,5-triazin-2-yl], or
Phenol,4,4′,4″,4′″-(1,2-ethanediylbis(imino-1,3,5-triazine-6,2,4-triylbis(thio)))tetrakis(2,6-bis(1,1-dimethyletyl)).

17. A composition comprising a compound of formula I or a stereoisomer, tautomer, racemic, metabolite, pro- or predrug, salt, hydrate, or solvate thereof, 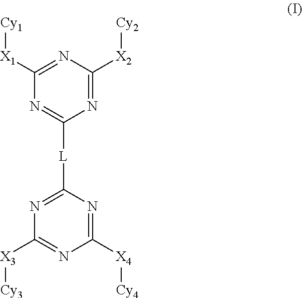
Wherein
X₁, X₂, X₃, and X₄ are independently selected from the group consisting of —NRₐ—, —O—, —S—, and —CHR_{b}—;
Rₐ and R_{b} are each independently selected from H and alkyl;
Cy₁, Cy₂, Cy₃ and Cy₄ are each phenyl; wherein said phenyl is optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group consisting of haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula 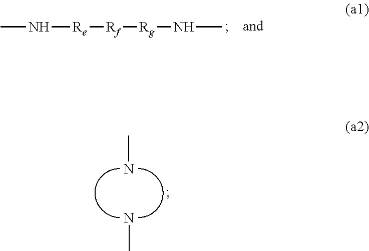
wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group consisting of aryl, heteroaryl, cycoalkyl, and heterocyclyl;
wherein —Rₑ—R_{f}—R_{g}— is not a direct bond; and wherein at least one of Cy₁, Cy₂, Cy₃, or Cy₄, is substituted with two or more substituents;
for use as a medicine.

18. A method for preventing or treating an infectious disease, said method comprising administering to a subject in need thereof a therapeutic effective amount of a compound of formula I or a stereoisomer, tautomer, racemic, metabolite, pro- or predrug, salt, hydrate, or solvate thereof, 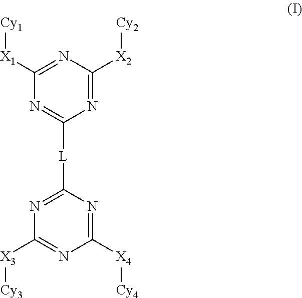
Wherein
X₁, X₂, X₃, and X₄ are independently selected from the group consisting of —NRₐ—, —O—, —S—, and —CHR_{b}—;
Rₐ and R_{b} are each independently selected from H and alkyl;
Cy₁, Cy₂, Cy₃ and Cy₄ are each phenyl; wherein said phenyl is optionally substituted with one or more substituents selected from -Alk and —R_{c};
wherein each -Alk is independently selected from alkyl, alkenyl, and alkynyl; wherein said alkyl, alkenyl, and alkynyl is optionally substituted with one or more —R_{d};
wherein each —R_{c} and —R_{d} is independently selected from the group consisting of haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula 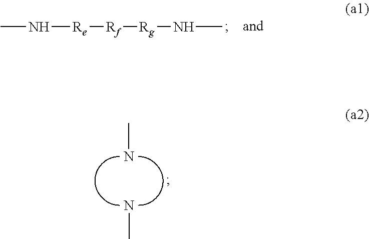
wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group consisting of aryl, heteroaryl, cycoalkyl, and heterocyclyl;
wherein —Rₑ—R_{f}—R_{g}— is not a direct bond.

19. A compound as defined in claim 16, wherein:
L is selected from the groups with subformula 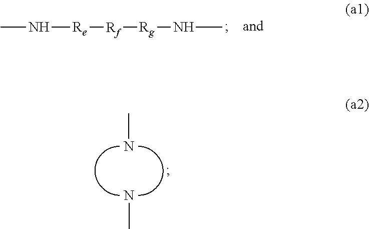
wherein subformula (a2) represents a 6-membered aromatic or non-aromatic heterocyclic group containing at least two nitrogen atoms; and
R_{f} is a direct bond or aryl.

20. A compound as defined in claim 16, wherein:
Rₐ and R_{b} are each independently selected from H and C₁₋₅alkyl;
Cy₁, Cy₂, Cy₃ and Cy₄ are each phenyl optionally substituted with one or more substituents independently selected from the group consisting of alkyl, alkoxy, halo, and cyano;
L is selected from the groups with subformula 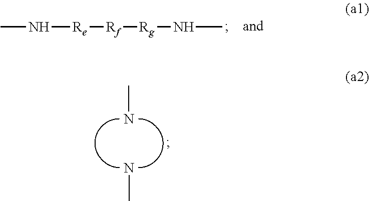
wherein subformula (a2) is pyrazinylene or piperazinylene; and
Rₑ and R_{g} are independently a direct bond or alkyl;
R_{f} is a direct bond or aryl.

21. A compound according to formula II 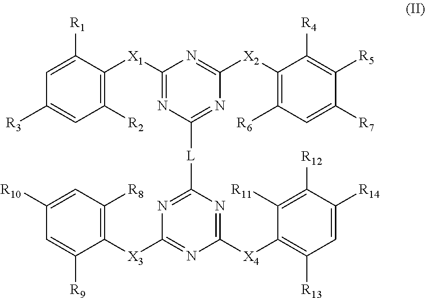
Wherein
X₁, X₂, X₃, and X₄ are independently selected from the group consisting of —NRₐ—, —O—, —S—, and —CHR_{b}—;
Rₐ and R_{b} are each independently selected from H and alkyl;
R₁, R₂, R₃, R₄, R₅, R₆, R₇, R₈, R₉, R₁₀, R₁₁, R₁₂, R₁₃, and R₁₄ are independently selected from the group consisting of H, alkyl, haloalkyl, alkoxy, haloalkoxy, hydroxy, halo, cyano, nitro, amino, —NHCOH, —C(═O)NH₂, —CH═CHCN, alkylamino-, dialkylamino-, and aminoalkyl-; and
L is selected from the groups with subformula 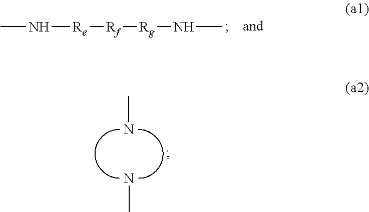
wherein subformula (a2) represents a 5- or 6-membered aromatic or non-aromatic heterocyclic group containing at least two nitrogen atoms;
Rₑ and R_{g} are independently a direct bond or alkyl; and
R_{f} is a direct bond or selected from the group consisting of aryl, heteroaryl, cycoalkyl, and heterocyclyl;
wherein at least one of the phenyl rings is substituted with two or more substituents other than hydrogen; and wherein —Rₑ—R_{f}—R_{g}— is not a direct bond,
provided that said compound is not
1,2-Ethanediamine, N,N′-bis[4,6-bis(2,6-dimethylphenoxy)-1,3,5-triazin-2-yl].

22. A compound according to claim 21, wherein:
X₁, X₂, X₃, and X₄ are independently selected from the group consisting of —NRₐ—, and —O—;
Rₐ and R_{b} are each independently selected from H and C₁₋₅alkyl;
R₁, R₂, R₃, R₄, R₅, R₆, R₇, R₈, R₉, R₁₀, R₁₁, R₁₂, R₁₃, and R₁₄ are independently selected from the group consisting of H, alkyl, alkoxy, halo, cyano, and —CH═CHCN; and
L is selected from the groups with subformula 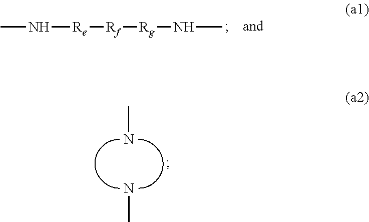
wherein subformula (a2) represents a 6-membered aromatic or non-aromatic heterocyclic group containing at least two nitrogen atoms;
Rₑ and R_{g} are independently a direct bond or C₁₋₅alkyl;
R_{f} is a direct bond or aryl;
wherein at least of one of the phenyl rings is substituted with two or more substituents other than hydrogen; and wherein —Rₑ—R_{f}—R_{g}— is not a direct bond,
provided that said compound is not
1,2-Ethanediamine, N,N′-bis[4,6-bis(2,6-dimethylphenoxy)-1,3,5-triazin-2-yl].

23. A composition comprising a compound according to claim 21, for use as a medicine.

24. A composition comprising a compound as defined in claim 21, for use as a human or veterinary medicine.

25. A method for preventing or treating an infectious disease, the method comprising administering to a subject in need thereof a therapeutic effective amount of a compound according to claim 21.

26. A method for preventing or treating a parasitic disease, the method comprising administering to a subject in need thereof a therapeutic effective amount of a compound as defined in claim 21.

27. A method for preventing or treating a parasitic disease, the method comprising administering to a subject in need thereof a therapeutic effective amount of a compound as defined in claim 21, wherein X₁ and X₃ represent —O—.

28. A method for preventing or treating a viral disease, the method comprising administering to a subject in need thereof a therapeutic effective amount of a compound as defined in claim 16.

29. A method for preventing or treating a viral disease, the method comprising administering to a subject in need thereof a therapeutic effective amount of a compound according to claim 21, wherein X₁ and X₃ represent —NH—.

30. A method for preventing or treating a disease associated with Trypanosoma, Leishmania, Plasmodium, or human immunodeficiency virus (HIV), the method comprising administering to a subject in need thereof a therapeutic effective amount of a compound as defined in claim 16.

31. A pharmaceutical composition comprising a compound as defined in claim 16 and one or more pharmaceutically acceptable carriers, diluents, excipients, or adjuvants.

32. A composition according to claim 17, further comprising one or more pharmaceutically acceptable carriers, diluents, excipients, or adjuvants.

33. A pharmaceutical composition comprising a compound as defined in claim 21 and one or more pharmaceutically acceptable carriers, diluents, excipients, or adjuvants.
